(19) United States
(12) Patent Application Publication
(10) Pub. No.: US 2014/0221827 A1
(43) Pub. Date: Aug. 7, 2014

(54) ENHANCED OPTICAL ANGIOGRAPHY USING INTENSITY CONTRAST AND PHASE CONTRAST IMAGING METHODS

(71) Applicants: S. M. Reza Motaghiannezam, Los Altos, CA (US); Scott E. Fraser, La Canada, CA (US)

(72) Inventors: S. M. Reza Motaghiannezam, Los Altos, CA (US); Scott E. Fraser, La Canada, CA (US)

(73) Assignee: CALIFORNIA INSTITUTE OF TECHNOLOGY, Pasadena, CA (US)

(21) Appl. No.: 14/124,206

(22) PCT Filed: Jun. 7, 2012
(86) PCT No.: PCT/US12/41403

§ 371 Date: Apr. 11, 2014

Related U.S. Application Data
(60) Provisional application No. 61/494,321, filed on Jun. 7, 2011.
(60) Provisional application No. 61/540,901, filed on Sep. 29, 2011.
(60) Provisional application No. 61/544,903, filed on Oct. 7, 2011.

(51) Int. Cl. G01B 9/02; A61B 5/00; A61B 5/11; A61B 3/00

(52) U.S. Cl. CPC G01B 9/02091; A61B 3/0025; A61B 5/0066; A61B 5/1128; A61B 5/489; A61B 5/4836; A61B 2576/00
USPC 600/425; 356/479; 351/206; 351/246

(57) ABSTRACT

The methods described herein are methods to ascertain motion contrast within optical coherence tomography data based upon intensity. The methods of the invention use logarithm operation to convert the multiplicative amplitude or intensity fluctuations (speckle) into the additive variations and recovers the motion contrasts by removing the speckle free signals (static regions) through statistical analysis.

### FIELD OF INVENTION

The invention provides various methods for ascertaining motion contrast in a sample. The embodiment of this invention describes methods to capture motion and generate motion contrast in an optical coherence tomography (OCT) system or other optical imaging systems (such as color fundus photography (CF), fluorescein angiography (FA), and indocyanine green angiography (ICGA)) by obtaining and analyzing data using the inventive methods based on statistical analysis of the logarithm intensities (or differences of logarithm intensities), joint statistical analysis of a function of phase differences and intensities (or intensity ratios), a combined statistical analysis of a function of phase differences and a function of intensities (or intensity ratios), or statistical analysis of a complex function of complex OCT signal ratios.

### BACKGROUND

There is a need for a simple OCT method that does not rely on the phase information and provides highly motion-sensitive contrast for distinguishing regions of motion from stationary areas. The latter is especially important for detecting leakage and abnormal vessels in patients with abnormal retinal and choroidal structure.

Further, in order to enhance the phase-based motion contrast methods such as differential phase variance (DPV) method, we develop joint statistical analysis of a function of phase differences and intensities, a function of intensity ratios and phase differences, or a complex function of complex OCT signal ratios. The proposed methods enhance contrast using extra information (a function of intensity, a function of intensity ratios).

In addition, CF, FA, ICGA methods are intensity-based methods and may not provide phase information of the back scattered light. While CF provides the structural information in the captured 2D en face view of retina, it may not identify the regions of motion in the 2D en face view. Thus, there is a need to enhance these intensity-based methods by adding the capability of motion detection to them. The proposed statistical analysis of the logarithm (or differences of logarithms) or ratios of the registered and captured 2D en face intensities (at different time points) is able to detect the regions of motion in 2D. The proposed methods may enhance contrasts in both FA and ICGA.

### BRIEF DESCRIPTION OF FIGURES

FIG. 1 illustrates a schematic diagram of an OCT system.

FIG. 2 illustrates a schematic diagram of the swept source (SS)-OCT used for all OCT data presented herein.

FIG. 3A illustrates a schematic of transverse scan patterns for one beam illumination.

FIG. 3B illustrates schematic of transverse scan patterns for multiple (two) beams illuminations.

FIG. 4 represents a flowchart of the OCT data processing procedures used for generating different motion contrast images.

FIG. 5 represents a flowchart of the data processing procedures used for generating four different motion contrasts including: (a) differential phase variance (DPV), (b) joint analysis of real and imaginary parts of the complex logarithm of complex OCT signals, (c) logarithmic intensity variance (LOGIV), and (d) differential logarithmic intensity variance (DLOGIV).

FIG. 6 represents a flowchart of the data processing procedures used for generalized intensity and differential phase contrast (GIDPC) imaging method (first approach-a).

FIG. 7 represents a flowchart of the data processing procedures used for generalized intensity and differential phase contrast (GIDPC) imaging method (second approach-b).

FIG. 8 represents a flowchart of the data processing procedures used for generalized intensity ratio and differential phase contrast (GIRDPC) imaging method (first approach-a).

FIG. 9 represents a flowchart of the data processing procedures used for generalized intensity ratio and differential phase contrast (GIRDPC) Imaging method (second approach-b).

FIG. 10 depicts a 2D OCT intensity tomogram across the fovea centralis (5 mm) in a normal subject's eye in vivo.

FIG. 11 depicts Foveal (a) average intensity, (b) speckle contrast ratio, (c) speckle variance, (d) LOGIV, (e) DLOGIV, (f) DPV before phase correction and compensation, and (g) DPV after phase timing induced phase error correction and bulk motion compensation tomograms (2 mm). White regions correspond to regions with higher either motion or/and reflectivity. White arrows indicate the small vessel in FIGS. 11(b)-11(g). IS/OS and RPE are located between two dashed lines and red boxes (static regions). White bands between two dotted lines and blue boxes indicate regions of motion in the inner choroid. One beam illumination method (N=4, T=5 ms, M=1) was employed for acquiring data as shown in FIG. 3(a). The same data processing procedures explained in FIGS. 4-5 were used.

FIG. 12 depicts parafoveal depth-integrated en face views over 4 mm² field of view (FOV) acquired in 4 seconds. Inverted (a) averaged intensity, (b) speckle contrast ratio, (c) speckle variance, (d) LOGIV, (e) DLOGIV, and (f) DPV (after phase correction and compensation) en face images of the inner retina. One beam illumination method (N=4, T=5 ms, M=200, OCT machine speed=50.4 kHz) was employed for acquiring data as shown in FIG. 3(a). The same data processing procedures explained in FIGS. 4-5 were used. In this figure, the rendering contrast is inverted so the highest intensity is shown in black to enable the smaller features to be more easily visualized.

FIG. 13 depicts parafoveal depth-integrated en face views over 4 mm² FOV acquired in 4 seconds. Inverted (a) LOGIV, (b) DLOGIV, and (c) DPV en face images of the retina between the regions 255 μm and 216 μm anterior to IS/OS. Inverted (d) LOGIV, (e) DLOGIV, and (f) DPV en face images of the retina between the regions 216 μm and 169 μm anterior to IS/OS. One beam illumination method (N=4, T=5 ms, M=200, OCT machine speed=50.4 kHz) was employed for acquiring data as shown in FIG. 3(a). The same data processing procedures explained in FIGS. 4-5 were used. In this figure, the rendering contrast is inverted so the highest intensity is shown in black to enable the smaller features to be more easily visualized.

FIG. 14 illustrates foveal depth-integrated JDIPC en face view over 4 mm² FOV acquired in 4 seconds depicting the inner plexiform and nuclear layers capillaries. The same data processing procedures explained in FIG. 4 and FIG. 5(b) were used. The covariance between real and imaginary parts were calculated (Eq. 7) for statistical analysis and capturing motion.

FIG. 15 illustrates foveal depth-integrated GIDPC (second approach-b) en face view over 4 mm² FOV acquired in 4 seconds depicting the inner plexiform and nuclear layers capillaries. The same data processing procedures explained in FIG. 4 and FIG. 7 were used, where G₁(x)=log(x) (Eq. 15), G₂(y)=y (Eq. 16), m=n=2, and K(a,b)=a+b (Eq. 17), respectively. The motion contrast is given by σ²_{log(I)}+σ²_{Δφ} as shown in Eq. 20.

FIG. 16 illustrates foveal depth-integrated GIRDPC (second approach-b) en face view over 4 mm² FOV acquired in 4 seconds depicting the inner plexiform and nuclear layers capillaries. The same data processing procedures explained in FIG. 4 and FIG. 9 were used, where G₁(x)=log(x) (Eq. 28), G₂(y)=y (Eq. 29), m=n=2, and K(a,b)=a+b (Eq. 30), respectively. The motion contrast is given by σ²_{Δ log (I)}+σ²_{Δφ} as shown in Eq. 33.

FIG. 17 depicts comparisons between proposed methods (LOGIV and DLOGIV) and FA. (a-b) FA images over scanning angles of 50°×50° in two normal subjects' right and left eyes. (c-d) FA images over scanning angles of 6°×6° in the same regions of normal subjects' right and left eyes (signified with white dashed line). Parafoveal (e-f) DLOGIV and (g) LOGIV OCT depth-integrated en face views of the retina between the regions 255 μm and 216 μm anterior to IS/OS over scanning angles of 6°×6° in the same signified areas in (a) and (b), respectively. DLOGIV (e) and LOGIV (g) en face images achieve the similar contrast for foveal vasculature visualization. Parafoveal (h) DLOGIV OCT depth-integrated en face views of the retina between the 216 μm and 169 μm anterior to IS/OS over scanning angles of 6°×6° in the same signified areas in (b). No foveal avascular zone (FAZ) is discernible in the normal subject-2 ((f-h)). (f) and (h) reveal depth-related variations of capillary meshwork morphology through the inner retina.

FIG. 18 depicts a flowchart representing the required procedures for vasculature visualization using logarithmic intensity method. Parafoveal en face view over 4 mm² FOV.

FIG. 19 depicts a flowchart representing the required procedures for vasculature visualization using differential logarithmic intensity method. Parafoveal en face view over 4 mm² FOV.

### DETAILED DESCRIPTION OF THE INVENTION

Several methods are described to ascertain motion contrast within optical coherence tomography (OCT) and optical imaging (such as color fundus photography (CF)). While the statistical analysis of the linear intensity may not differentiate regions of motion from stationary regions, the statistical analysis of an optimized function of linear intensities such as logarithm intensities provides a surrogate marker for motion. The inventive OCT methods of calculating motion contrast from the logarithm intensities (or differences of logarithm intensities) can differentiate regions of motion from static regions through depth and provide a 3D motion contrast image. The inventive CF methods of calculating motion contrast from the logarithm intensity (or differences of logarithm intensities) can differentiate regions of motion from static regions and provide a 2D (fundus) motion contrast image. The other methods improve contrast by using joint statistical analysis of a function of phase differences and intensities (or intensity ratios).

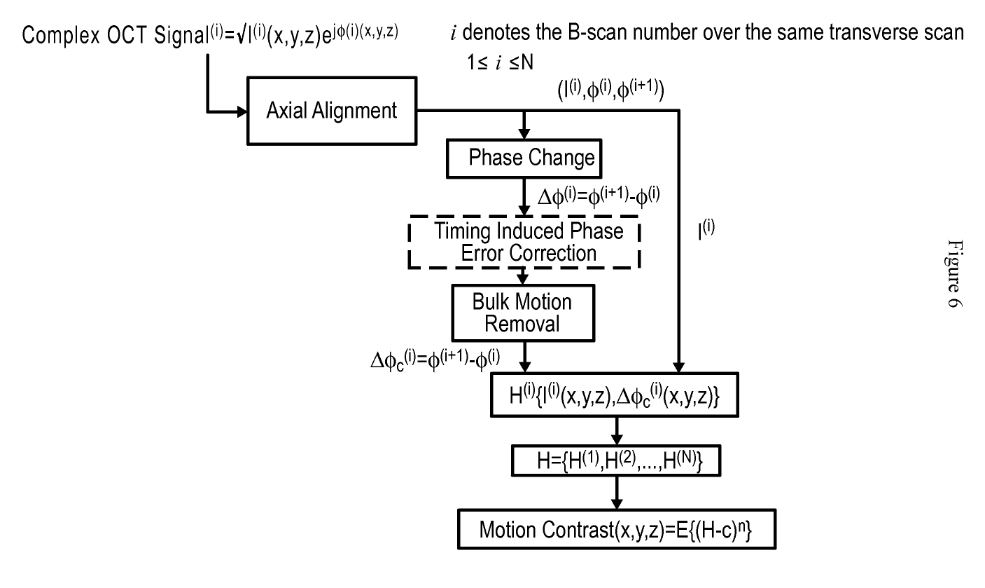

We test different approaches including: statistical analysis of (i) logarithm of intensity of OCT signals (FIG. 5c), (ii) differences between successive logarithm intensities of OCT signals (FIG. 5d), and (iii) differences between successive complex logarithms of complex OCT signals (FIG. 5b). Application of LOGIV, DLOGIV, and speckle contrasts (speckle variance and speckle contrast ratio) for 3D microvasculature imaging in the in vivo human retina is validated by employing a high-speed SS-OCT at 1060 nm. LOGIV and DLOGIV retinal en face views show the enhanced motion contrasts in comparison with speckle contrasts (such as speckle variance and speckle contrast ratio) for capturing microvasculature that lies between hyper-reflective regions. Compared to the differential phase variance (DPV) method (FIG. 5a), these logarithmic intensity-based motion contrast methods are simpler, have similar performance, and do not require extra software and hardware.

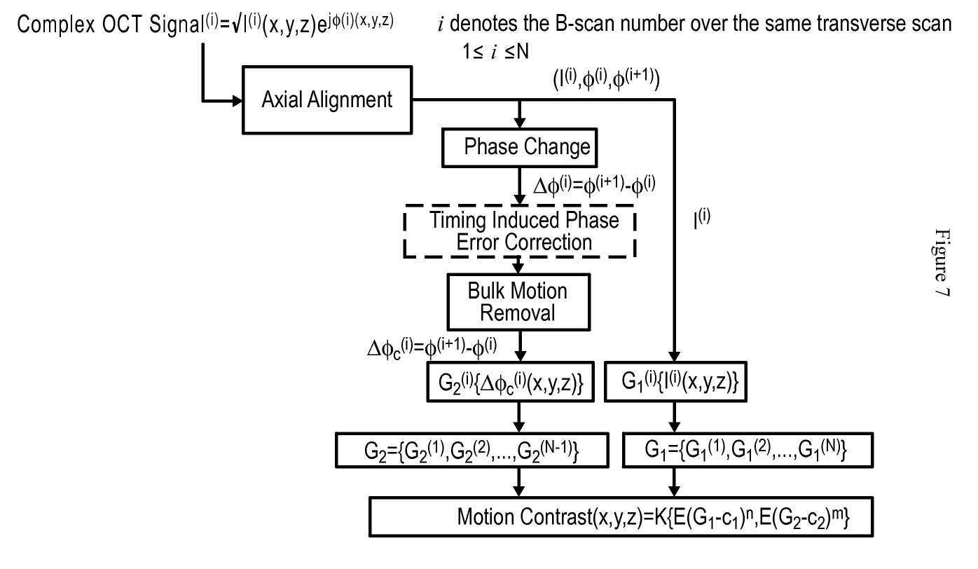

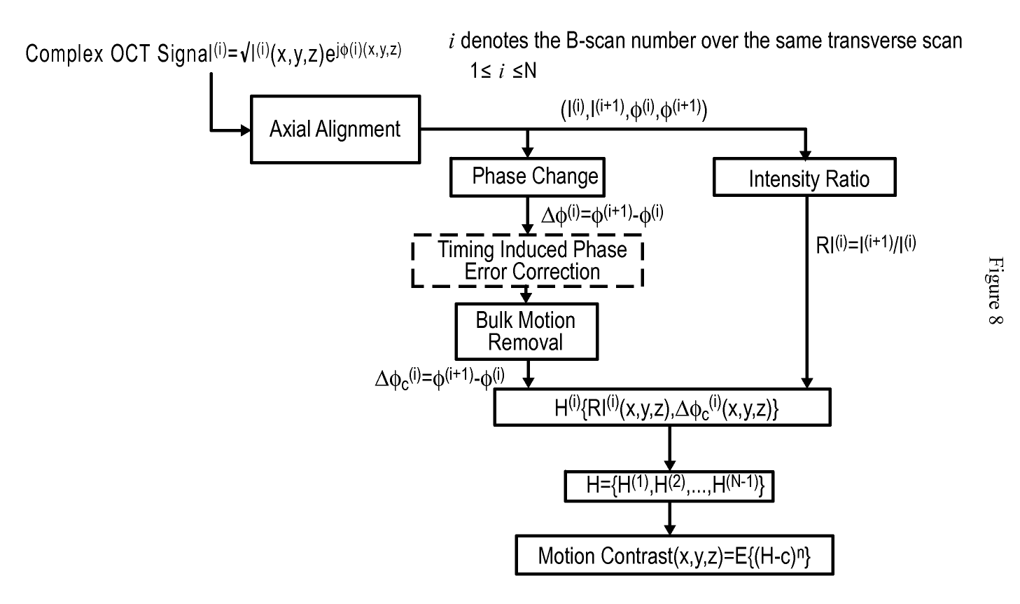

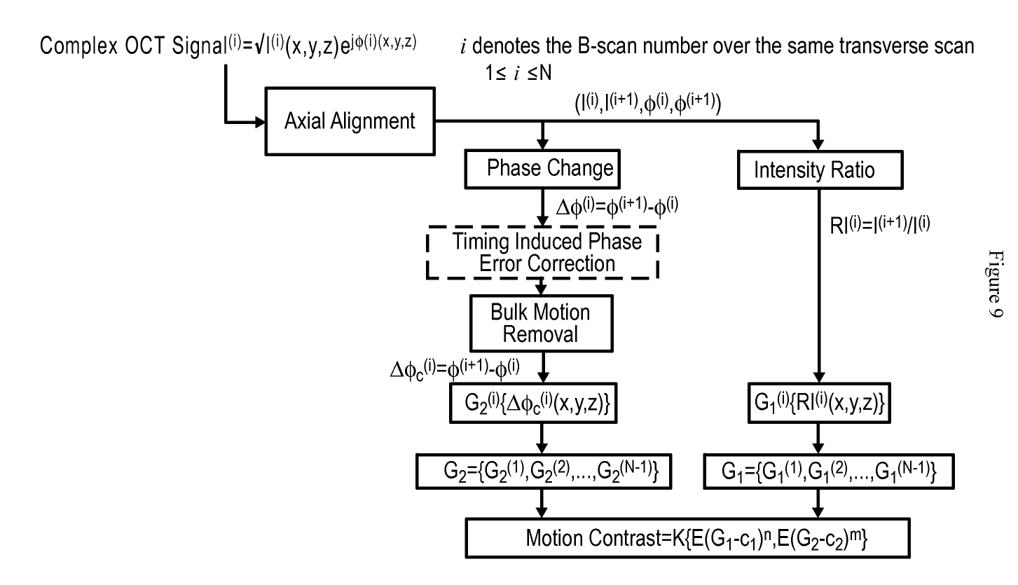

To generalize the abovementioned logarithmic motion contrasts and enhance them, we also purpose several motion-sensitive contrasts including: 1—statistical analysis of a function of linear intensities and phase differences of OCT signals (FIG. 6), 2—a function of two statistical measures of two independent functions of OCT intensities and phase differences (FIG. 7), 3—statistical analysis of a function of successive OCT intensity ratios and phase differences (FIG. 8), 4—a function of two statistical measures of two independent functions of successive OCT intensity ratios and phase differences (FIG. 9), and 5—a function of two statistical measures of two independent functions of magnitude and angle of successive complex OCT signal ratios.

The joint statistical analysis of any (nonlinear) function of (a) phase differences and linear (differences of) intensities of OCT signal, (b) complex OCT signals, and (c) ratios of successive complex OCT signals increases the number of independent random variables by a factor of two and improves motion contrast in comparison with other motion contrast method using a random variable such as differential phase variance (DPV) method.

Accordingly, the invention provides various methods for detecting motion in a sample. The method comprises ascertaining motion contrast in the sample according to the methods described below and detecting the motion in the sample based on the motion contrast.

The invention is directed to a method for ascertaining motion contrast in a sample using an optical coherence tomography (OCT) system. The method comprises (i) acquiring multiple B-scans of the sample separated in time over the same transverse position using OCT, wherein each of the B-scans comprises data acquired during multiple A-scans over a range of transverse locations, (ii) acquiring multiple OCT intensity (I) measurements based on the data of the B-scans over the same transverse point separated in time, (iii) ascertaining logarithms of the OCT intensity measurements over the same transverse point separated in time, (iv) ascertaining motion contrast based upon the variance of logarithmic intensity measurements of the same transverse point acquired in the successive B-scans separated in time, and (v) repeating the same described procedures (i-iv) for the adjacent transverse points in the same and neighboring B-scans to ascertain motion contrast in the sample. In one embodiment, motion contrast based on the variance of the measured logarithm intensities (FIG. 5c) in the successive B-scans is ascertained according to Equation 2. In another embodiment, motion contrast based on the variance of differences of the logarithm intensities (FIG. 5d) between the successive B-scans is ascertained according to Equation 4. In an additional embodiment, the variance of logarithm intensity is ascertained independent of OCT phase data.

The invention further provides a method (FIG. 5b) for ascertaining motion contrast in a sample, comprising (i) acquiring multiple B-scans separated in time over the same transverse position using OCT, (ii) acquiring multiple complex OCT signals based on the B-scans over the same transverse point separated in time, (iii) ascertaining complex logarithms of the complex OCT signals over the same transverse point separated in time, (iv) ascertaining differences between the successive calculated complex logarithms for the same transverse point, (v) ascertaining the statistical measure between the real and corrected and compensated imaginary parts of the complex logarithm differences for the same transverse point, (vi) ascertaining the motion contrast based on the calculated statistical measure, and (vii) repeating the same described procedures (i-vi) for the adjacent transverse points in the same and neighboring B-scans to ascertain motion contrast in the sample. In some embodiments, the complex OCT signals based on the B-scans are acquired according to Equation 1, the complex logarithms of the complex OCT signals based on the B-scans are ascertained according to Equation 5, the differences between the corrected and compensated complex logarithms are ascertained according to Equation 6 and the motion contrast is ascertained according to Equation 7.

The invention provides an additional method (FIG. 6) for ascertaining motion contrast in a sample using an OCT system, comprising (i) acquiring multiple B-scans separated in time over the same transverse position using OCT, (ii) acquiring multiple OCT intensity (I) measurements based on the B-scans over the same transverse point separated in time, (iii) acquiring multiple OCT phase measurements based on the B-scans over the same transverse point separated in time, (iv) ascertaining corrected and compensated differences between the successive OCT phase measurements (Δφ) for the same transverse point separated in time, (v) ascertaining a variable h according to: h=H(I,Δφ); where H denotes a function I and Δφ, (vi) ascertaining a n^{th} moment of the variable h about a deterministic value of c, wherein n is an integer, (vii) ascertaining the motion contrast based on the n^{th} moment, and (viii) repeating the same described procedures for the adjacent transverse points in the same and neighboring B-scans to ascertain motion contrast in the sample. In some embodiments, the deterministic value of c is the mean of h, n=2, H(a,b)=log(a)+b and the motion contrast is ascertained according to Equation 14.

The invention further provides a method (FIG. 7) for ascertaining motion contrast in a sample, comprising (i) acquiring multiple B-scans separated in time over the same transverse position using OCT, (ii) acquiring multiple OCT intensity measurements (I) based on the B-scans over the same transverse point separated in time, (iii) ascertaining a variable g₁ according to: g₁=G₁(I); where G₁ denotes a function of variable I, (iv) ascertaining a n^{th} moment of the variable g₁ about a deterministic value of c₁, wherein n is an integer, (v) acquiring multiple OCT phase measurements based on the B-scans over the same transverse point separated in time, (vi) ascertaining corrected and compensated differences between the OCT phase measurements (Δφ) for the same transverse point separated in time, (vii) ascertaining a variable g₂ according to: g₂=G₂(Δφ_{c}); where G₂ denotes a function of Δφ_{c}, (viii) ascertaining a m^{th} moment of the variable g₂ about a deterministic value of c₂, wherein m is an integer, (ix) ascertaining a variable k according to: k=K(n^{th} moment of the variable g₁ about a deterministic value of c₁, m^{th} moment of the variable g₂ about a deterministic value of c₂), wherein m and n are integers and K denotes a function of two variables, (x) ascertaining the motion contrast based on the variable k, and (xi) repeating the same described procedures for the adjacent transverse points in the same and neighboring B-scans to ascertain motion contrast in the sample. In some embodiments of this method, G₁(x)=log(x), G₂(y)=y, n=m=2, the deterministic values of c₁ and c₂ are the mean of g₁ and g₂, respectively, k=K(a,b)=a+b; and the motion contrast is ascertained according to Equation 20.

The invention also provides a method (FIG. 8) for ascertaining motion contrast in a sample, comprising (i) acquiring multiple B-scans separated in time over the same transverse position using OCT, (ii) acquiring multiple OCT intensity (I) measurements based on the B-scans over the same transverse point separated in time, (iii) ascertaining linear intensity ratios (RIs) between the successive OCT intensity measurements for the same transverse point, (iv) acquiring multiple OCT phase measurements based on the B-scans over the same transverse point separated in time, (v) ascertaining corrected and compensated differences between the successive OCT phase measurements (Δφ) for the same transverse point separated in time, (vi) ascertaining a variable h according to: h=H(RI, Δφ_{c}); where H denotes a function of RI and Δφ_{c}, (vii) ascertaining a n^{th} moment of the variable h about a deterministic value of c, wherein n is an integer, (viii) ascertaining the motion contrast based on the n^{th} moment, and (ix) repeating the same described procedures for the adjacent transverse points in the same and neighboring B-scans to ascertain motion contrast in the sample. In some embodiments, the deterministic value of c is the mean of h, n=2, H(a,b)=log(a)+b and the motion contrast is ascertained according to Equation 27.

The invention provides a further method (FIG. 9) for ascertaining motion contrast in a sample, comprising (i) acquiring multiple B-scans separated in time over the same transverse position using OCT, (ii) acquiring multiple OCT intensity measurements (I) based on the B-scans over the same transverse point separated in time, (iii) ascertaining linear intensity ratios (RIs) between the successive OCT intensity measurements for the same transverse point, (iv) ascertaining a variable g₁ according to: g₁=G₁(RI); where G₁ denotes a function of variable RI, (v) ascertaining a n^{th} moment of the variable g₁ about a deterministic value of c₁, wherein n is an integer, (vi) acquiring multiple OCT phase measurements based on the B-scans over the same transverse point separated in time, (vii) ascertaining corrected and compensated differences between the OCT phase measurements (Δφ) for the same transverse point separated in time, (viii) ascertaining a variable g₂ according to: g₂=G₂(Δφ_{c}); where G₂ denotes a function of variable Δφ_{c}, (ix) ascertaining a m^{th} moment of the variable g₂ about a deterministic value of c₂, wherein m is an integer, (x) ascertaining a variable k according to: k=K(n^{th} moment of the variable g₁ about a deterministic value of c₁, m^{th} moment of the variable g₂ about a deterministic value of c₂), wherein m and n are integers and K denotes a function of two variables, (xi) ascertaining the motion contrast based on the variable k, and (xii) repeating the same described procedures for the adjacent transverse points in the same and neighboring B-scans to ascertain motion contrast in the sample. In some embodiments of this method, G₁(x)=log(x), G₂(y)=y, n=m=2, the deterministic values of c₁ and c₂ are the mean of g₁ and g₂, respectively, k=K(a,b)=a+b, and the motion contrast is ascertained according to Equation 33.

Also provided is a method for ascertaining motion contrast in a sample, comprising (i) acquiring multiple B-scans separated in time over the same transverse position using OCT, (ii) acquiring multiple complex OCT signals based on the B-scans over the same transverse point separated in time, (iii) ascertaining complex OCT signal ratios (RCSs) between the successive OCT signal measurements for the same transverse point, (iv) ascertaining a variable g1 according to: g₁=G₁(abs(RCS)); where G₁ denotes a function of variable of abs(RCS), (v) ascertaining a n^{th} moment of the variable g₁ about a deterministic value of c₁, wherein n is an integer, (vi) ascertaining a variable g₂ according to: g₂=G₂ (corrected and compensated angle (RCS) where G₂ denotes a function of corrected and compensated variable of angle (RCS), (viii) ascertaining a m^{th} moment of the variable g₂ about a deterministic value of c₂, wherein m is an integer, (ix) ascertaining a variable k according to: k=K(n^{th} moment of the variable g₁ about a deterministic value of c₁, m^{th} moment of the variable g₂ about a deterministic value of c₂), wherein m and n are integers and K denotes a function of two variables, (x) ascertaining the motion contrast based on the variable k, and (xi) repeating the same described procedures for the adjacent transverse points in the same and neighboring B-scans to ascertain motion contrast in the sample. In some embodiments of this method, G₁(x)=log x, G₂(y)=y, n=m=2, the deterministic values of c₁ and c₂ are the mean of g₁ and g₂, respectively, k=K(a,b)=a+b and the motion contrast is ascertained according to Equation 33.

In various embodiments of the methods described above, the motion contrast is ascertained by acquiring multiple B-scans separated in time using either a beam illumination in the sample arm of OCT system which scans the same transverse position multiple times (FIG. 3a) or multiple coded frequency or polarization beam illuminations separated in time in the sample arm of a single or multiple OCT system which scan the same transverse position one (or multiple) times (FIG. 3b).

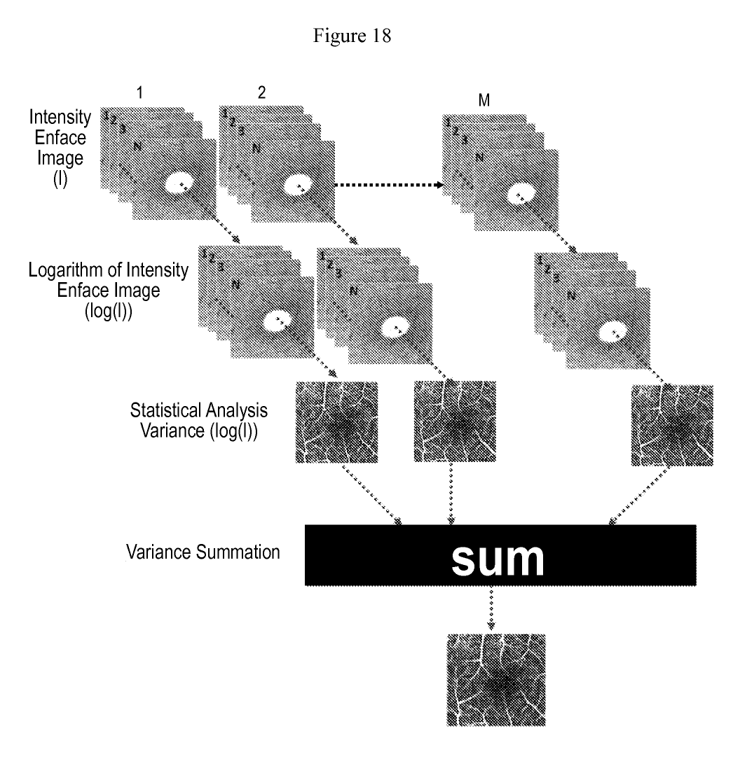

The invention also provides a method (FIG. 18) for ascertaining motion contrast in a sample based on images acquired using a digital camera. The method comprises (i) acquiring a set of N images of the sample using a digital camera and fundus illuminator, (ii) acquiring a set of N intensity measurements (I) based on the set of N images, (iii) ascertaining a set of N logarithms (log I) based on the set of N intensity measurements, (iv) ascertaining a n^{th} moment of the set of N logarithms about a deterministic value of c, and (v) ascertaining the motion contrast based on the n^{th} moment, wherein n and N are integers. In some embodiments, the deterministic value of c is the mean of the set of N logarithms, the n^{th} moment=E{[log(I)−c]ⁿ} and the motion contrast is ascertained according to Equation 35 for n=2. In one embodiment, the digital camera is a charge coupled device (CCD). In another embodiment, the digital camera is a complementary metal oxide semiconductor (CMOS) camera. The same method may be applicable for FA and ICGA.

The invention further provides a method (FIG. 18) for ascertaining motion contrast in a sample, comprising (i) acquiring a set of N images of the sample using a digital camera and fundus illuminator, (ii) acquiring a set of N intensity measurements (I) based on the set of N images, (iii) ascertaining a set of N logarithms (log I) based on the set of N intensity measurements, (iv) ascertaining a n^{th} moment of the set of N logarithms about a deterministic value of c, (v) acquiring M n^{th} moments by repeating the steps of (i)-(iv) M times, and (vi) ascertaining the motion contrast based on the sum of the M n^{th} moments, wherein M, N and n are integers. In some embodiments, the deterministic value of c is the mean of the set of N logarithms, the n^{th} moment=E{[log(I)−c]ⁿ} and the motion contrast is ascertained according to Equation 36 for n=2. In one embodiment, the digital camera is a charge coupled device (CCD). In another embodiment, the digital camera is a complementary metal oxide semiconductor (CMOS) camera. The same method may be applicable for FA and ICGA.

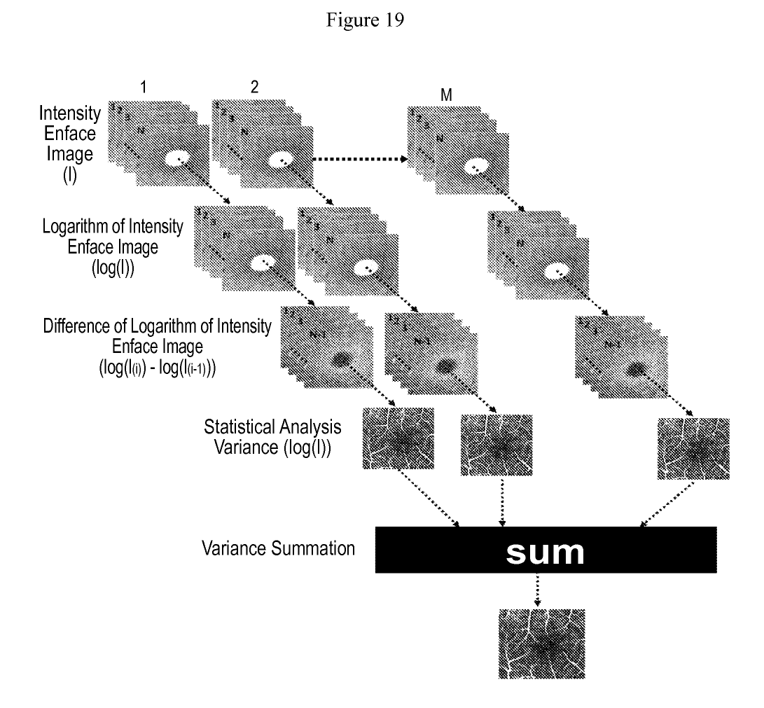

The invention also provides a method (FIG. 19) for ascertaining motion contrast in a sample, comprising (i) acquiring a set of N images of the sample using a digital camera and fundus illuminator, (ii) acquiring a set of N intensity measurements (I) based on the set of N images, (iii) ascertaining a set of N logarithms (log I) based on the set of N intensity measurements, (iv) ascertaining a set of N−1 logarithm differences (Δ log I) between two successive logarithms based on the set of N logarithms, (v) ascertaining a n^{th} moment of the set of N−1 logarithm differences about a deterministic value of c, and (vi) ascertaining the motion contrast based on the n^{th} moment, wherein n and N are integers. In some embodiments of this methods, the deterministic value of c is the mean of the set of N−1 logarithm differences, the n^{th} moment=E{[Δ log(I)−c]ⁿ} and the motion contrast is ascertained according to Equation 38 for n=2. In one embodiment, the digital camera is a charge coupled device (CCD). In another embodiment, the digital camera is a complementary metal oxide semiconductor (CMOS) camera. The same method may be applicable for FA and ICGA.

The invention further provides a method (FIG. 19) for ascertaining motion contrast in a sample, comprising (i) acquiring a set of N images of the sample using a digital camera and fundus illuminator, (ii) acquiring a set of N intensity measurements (I) based on the set of N images, (iii) ascertaining a set of N logarithms (log I) based on the set of N intensity measurements, (iv) ascertaining a set of N−1 logarithm differences (Δ log I) between two successive logarithms based on the set of N logarithms, (v) ascertaining a n^{th} moment of the set of N−1 logarithm differences about a deterministic value of c, (vi) acquiring M n^{th} moments by repeating the steps of (i)-(v) M times, and (vii) ascertaining the motion contrast based on the sum of the M n^{th} moment, wherein M, N and n are integers. In some embodiments of this methods, the deterministic value of c is the mean of the set of N−1 logarithm differences, the n^{th} moment=E{[Δ log(I)−c]ⁿ} and the motion contrast is ascertained according to Equation 39 for n=2. In one embodiment, the digital camera is a charge coupled device (CCD). In another embodiment, the digital camera is a complementary metal oxide semiconductor (CMOS) camera. The same method may be applicable for FA and ICGA.

The invention also provides a method for ascertaining motion contrast in a sample, comprising (i) acquiring a set of N images of the sample using a digital camera and fundus illuminator, (ii) acquiring a set of N intensity measurements (I) based on the set of N images, (iii) ascertaining a set of N−1 intensity ratios (RI) between two successive intensity measurements based on the set of N intensity measurements, (iv) ascertaining a n^{th} n moment of the set of N−1 intensity ratios about a deterministic value of c, and (v) ascertaining the motion contrast based on the n^{th} moment, wherein n and N are integers. In some embodiments, the deterministic value of c is the mean of the set of N−1 intensity ratios and the n^{th} moment=E{[RI−c]ⁿ}. In one embodiment, the digital camera is a charge coupled device (CCD). In another embodiment, the digital camera is a complementary metal oxide semiconductor (CMOS) camera. The same method may be applicable for FA and ICGA.

Additionally a method for ascertaining motion contrast in a sample comprises (i) acquiring a set of N images of the sample using a digital camera and fundus illuminator, (ii) acquiring a set of N intensity measurements (I) based on the set of N images, (iii) ascertaining a set of N−1 intensity ratios (RI) between two successive intensity measurements based on the set of N intensity measurements, (iv) ascertaining a n^{th} moment of the set of N−1 intensity ratios about a deterministic value of c, (v) acquiring M n^{th} n moments by repeating the steps of (i)-(iv) M times, and (vi) ascertaining the motion contrast based on the sum of the M n^{th} moment, wherein n, N and M are integers. In some embodiments, the deterministic value of c is the mean of the set of N−1 intensity ratios and the n^{th} moment=E{[RI−c]ⁿ}. In one embodiment, the digital camera is a charge coupled device (CCD). In another embodiment, the digital camera is a complementary metal oxide semiconductor (CMOS) camera. The same method may be applicable for FA and ICGA.

The invention further provides methods for diagnosing/treating a disease in an individual. The methods comprise detecting motion contrast in an area of the individual according to any of the methods described above and diagnosing/treating the disease in the individual based on the detected motion. Examples of diseases that may be diagnosed based on the methods described herein include but are not limited to various eye diseases, such as diabetic retinopathy, age-related macular degeneration (AMD), glaucoma and anterior ischemic optic neuropathy (AION).

The invention further provides methods for visualizing vasculature in a sample. The method comprises ascertaining motion contrast in the sample according to the methods described above and visualizing the vasculature based on the motion contrast.

Also provided is a computer readable medium having computer executable instructions for ascertaining motion contrast in a sample according to any of the method described above. Also provided is an OCT system comprising a computer readable medium having computer executable instruction for ascertaining motion contrast in a sample according to any of the methods described above.

### Advantages of the Invention

Speckle variance vascular visualization has been reported by applying variance to the linear intensity of the received OCT intensity signal. This method captures motion through analyzing the temporal linear intensity fluctuation. However, this method highlights not only the regions of motion but also hyper-reflective stationary regions. To remove the direct dependence of the speckle on the sample reflectivity (such as hyper-reflective regions), statistical analysis of a natural logarithm of OCT intensities is described. The proposed logarithm operation converts the multiplicative amplitude or intensity fluctuations (speckle) into the additive variations and recovers the motion contrasts by removing the speckle free signals (static regions) through statistical analysis. The logarithmic motion contrast methods enhance motion contrast by degrading variance of hyper-reflective stationary regions such as retina pigment epithelium (RPE). These methods can be also applied to other linear intensity-based contrast imaging methods such as optical microvasculature angiography (OMAG) to enhance contrast by removing stationary layers with high reflectivity.

### EXAMPLES

### Experimental Setup

The experimental methods described herein are applicable to all the examples described below, as appropriate.

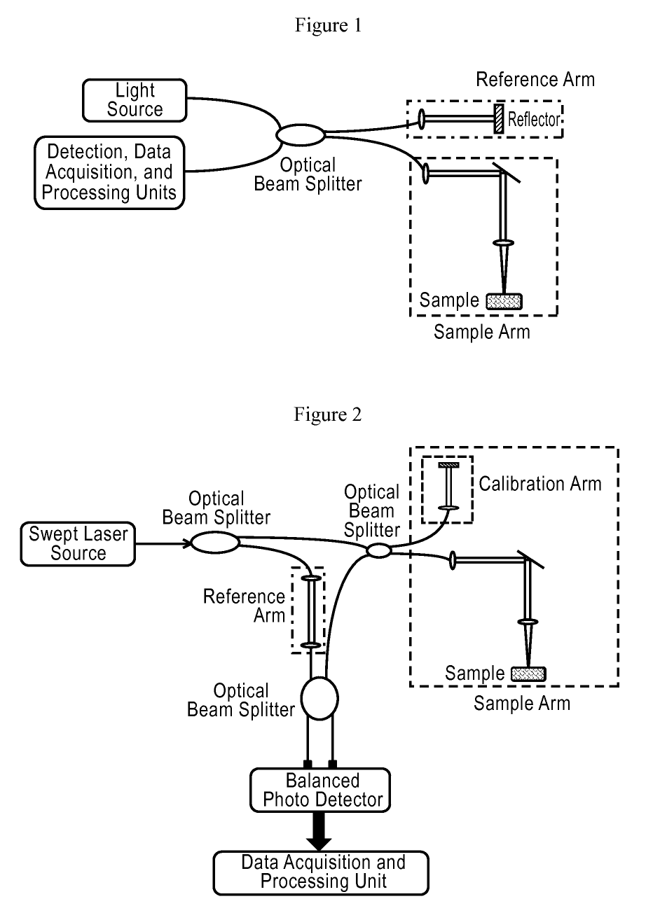

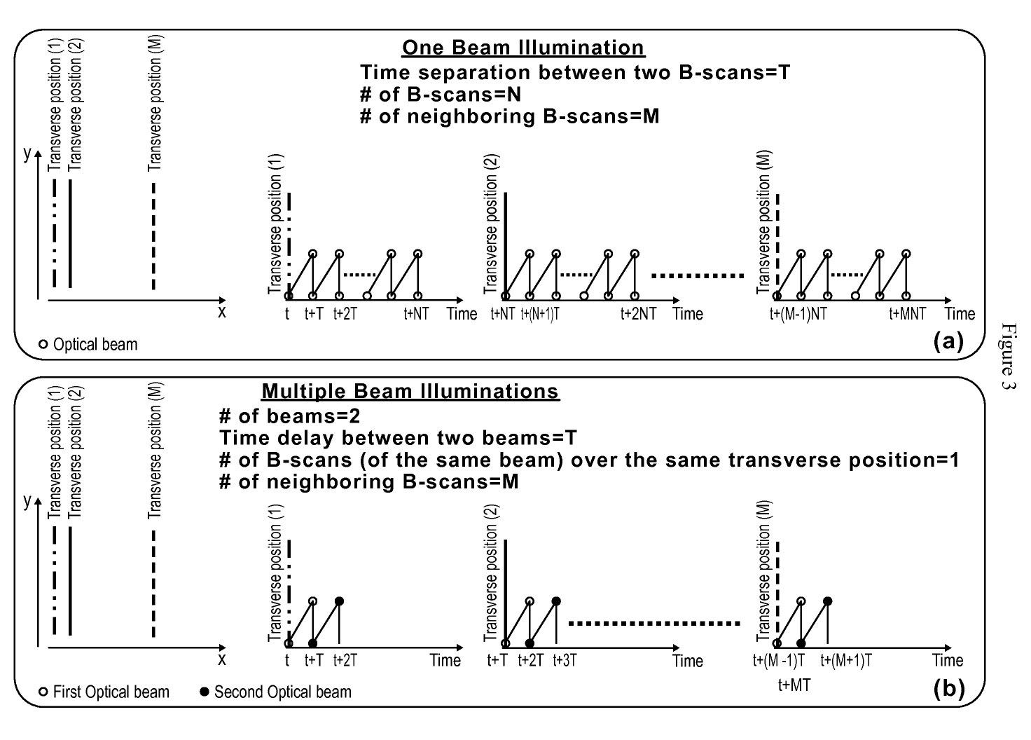

A schematic diagram of an OCT system (time domain/spectral domain/Fourier domain) was depicted in FIG. 1. To validate the proposed methods for providing motion contrasts and compare them with each other, we used a prototype 50.4 kHz phase sensitive SS-OCT system, incorporating a polygon-based 1060 nm (1015-1103) swept laser source, with ˜5.9 μm axial resolution in tissue and 102 dB sensitivity (1.2 mW incident power). The SS-OCT system was comprised of the polygon-based swept-laser source, an interferometer, and a data acquisition (DAQ) unit (FIG. 2). The swept source output was coupled to the interferometer through an isolator where a 90/10 coupler was used to split light into a sample arm: reference arm. The sample arm light was split equally between the calibration arm and a slit lamp biomicroscope as shown in FIG. 2. A 50/50 coupler combined and directed the reflected light from the sample to the one port of the interferometer output coupler. The reference arm light passed through a pair of collimators and was directed to the second port of the interferometer output coupler. The resulting interference fringes were detected on both output ports using a dual balanced photodetector. The spectral signals were continuously digitized by triggering an AD conversion board. A D/A board was used to generate the driving signals of the two-axis galvanometers. A user interface and data acquisition was developed in LabView to coordinate instrument control and enable user interaction.

### Scanning Protocols

The prototype SS-OCT instrument was used to image four eyes of two healthy volunteers. Total exposure time and incident exposure level were kept less than 5.5 seconds and 1.2 mW in each imaging session, consistent with the safe exposure determined by American National Standards Institute (ANSI) and International Commission on Non-Ionizing Radiation Protection (ICNIRP). In patient interface, a 60-D lens was used to provide a beam diameter of 1.5 mm on the cornea (˜15 μm transverse resolution).

Two illumination methods are able to capture the proposed motion contrasts including: (a) one beam illumination (FIG. 3(a)) and (b) multiple beam illuminations (FIG. 3(b)). The first illumination method was implemented for all the captured motion contrast results. Two scanning protocols were implemented. A 2D protocol acquired four horizontal tomograms (B-scans) with 201 depth scans (A-scans) spanning the same transverse slice (2 mm) across the foveal centralis in 0.02 seconds. In the second protocol, a 3D OCT data set was collected by acquiring several neighboring B-scans over the parafovea. The system magnification, SS-OCT speed (50400 Hz), speed of the fast scan axis (200 Hz, T=5 ms) with fly-back time (1 ms), and data acquisition time (4 seconds) gave an image size of 201×200 pixels over a 2 mm×2 mm field of view (FOV); each B-scan was repeated four times (N=4). In the 3D scanning protocol, the fast scan axis was sagittal (superior-inferior) and the slow axis was horizontal (nasal-temporal). FIG. 3(a) depicts the second scanning protocol with N=4, T=5 ms, and M=200. In FIG. 3 (a-b), the fly back time was zero.

### Image Processing and Motion Contrast Imaging

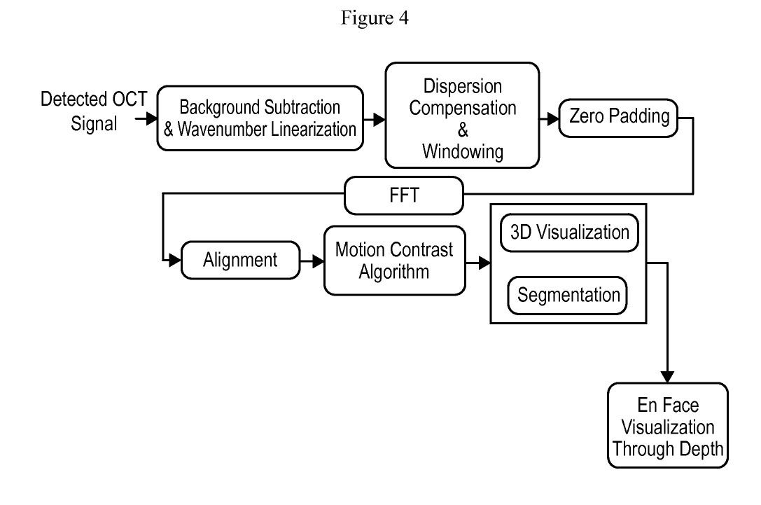

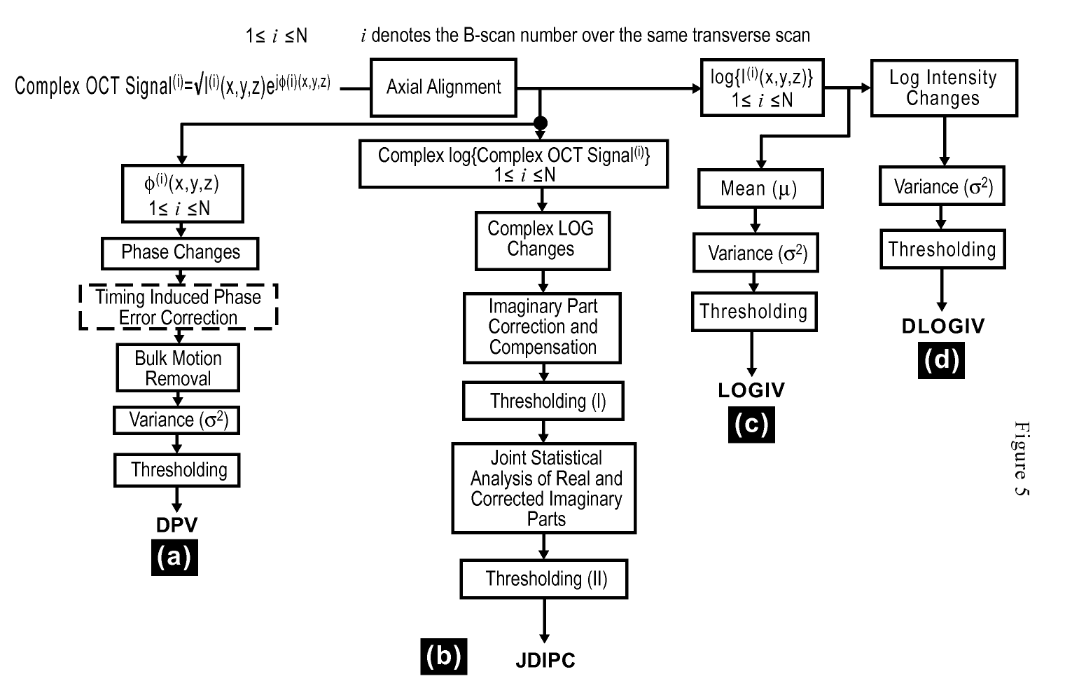

The digitized signals were divided into individual spectral sweeps in the post-processing algorithm (FIG. 4). Equal sample spacing in wave number (k) was achieved using a calibration trace at 1.5 mm interferometer delay and numerical correction of the nonlinearly swept waveforms. Image background subtraction and numeric compensation for second order dispersion were performed. The SS-OCT data sets were upsampled by a factor of 4 and Fourier transformed. Axial motion correction was achieved on the obtained 2D and 3D SS-OCT data sets by cross correlating the consecutive horizontal tomograms. The motion contrasts were calculated for all voxels through acquired depth scans. 3D motion contrast visualization was achieved by repeating the same procedure on the neighboring B-scans. For en face visualization, a segmentation algorithm was used and the calculated motion contrasts were summed over the desired depth.

### Motion Contrast Analysis and Imaging

To perform motion contrast analysis and imaging, four B-scans were acquired over the same transverse position (or slice). Time separations was T_{B}=5 ms between B-scans for capturing the same position, respectively. Multiple linear intensity and phase measurements were recorded over the same transverse point separated in time. Four different intensity-based approaches were tested: speckle variance, speckle contrast ratio, LOGIV, and DLOGIV.

In the speckle variance (σ²) and speckle contrast ratio (σ/μ) methods, the estimated linear intensity means (μ), variances (σ²) as well as the ratios between their estimated standard deviations and means (σ/μ) were calculated for the same transverse point acquired in successive B-scans. LOGIV was realized by calculating the estimated variance of multiple logarithmic intensity measurements (LOG(I(z,T))) of the same transverse point acquired in successive B-scans separated in time. DLOGIV and DPV captured the differences between multiple logarithmic intensity (LOG(I(z,T))) and phase measurements (φ(z,T)) of the same transverse points (separated in time) and calculated the estimated variance of these changes, respectively. To measure and remove timing-induced phase error due to the random delay between the trigger signal and the subsequent A-to-D conversion (sample clock), a calibration signal was generated using a stationary mirror in the calibration arm (FIG. 2). The calibration signal was located at a depth of 2 mm in the OCT intensity image. The corrected phase differences between adjacent B-scans for the same transverse point at a given depth were calculated by subtracting the phase difference of the calibration signal, linearly scaled with the sample signal depth, from the measured phase differences. Phase unwrapping was performed on all measurements. A weighted mean algorithm estimated and removed the bulk axial motion phase change error.

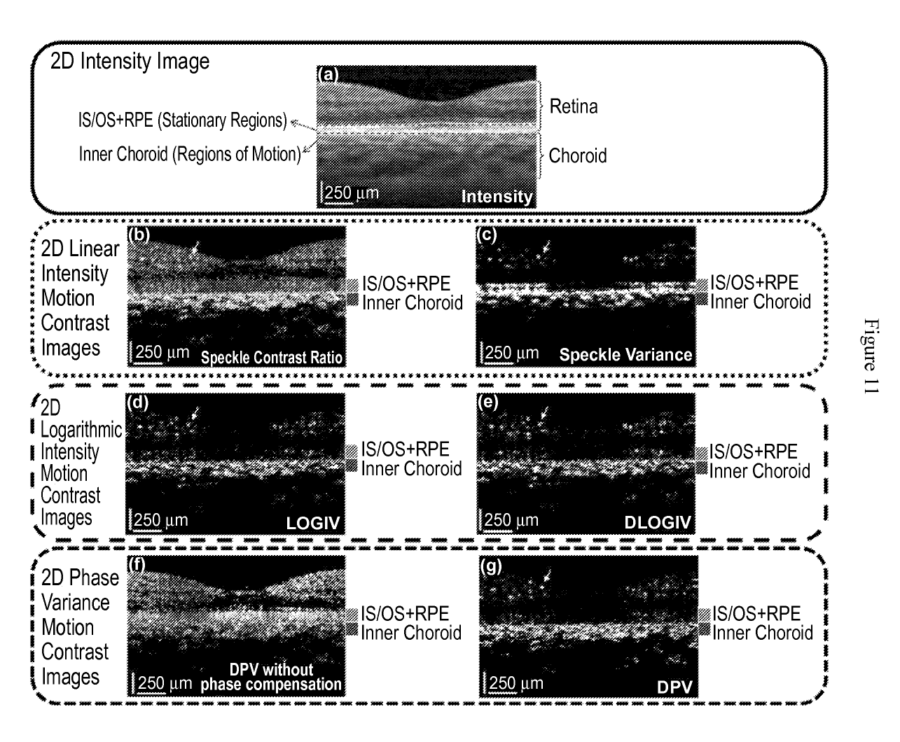

The same described procedures were repeated for the adjacent transverse points in the same and neighboring B-scans to capture the retinal vasculature in 2D and 3D data sets. To remove SNR-limited intensity and phase change errors in 2D and 3D data sets for vasculature visualization, an average intensity threshold (10 dB above the mean value of the noise floor) was applied; all contrasts with average intensity values<mean (log₁₀(Iₙₒᵢₛₑ))+10 dB were set to zero in the corresponding images (FIGS. 11-17).

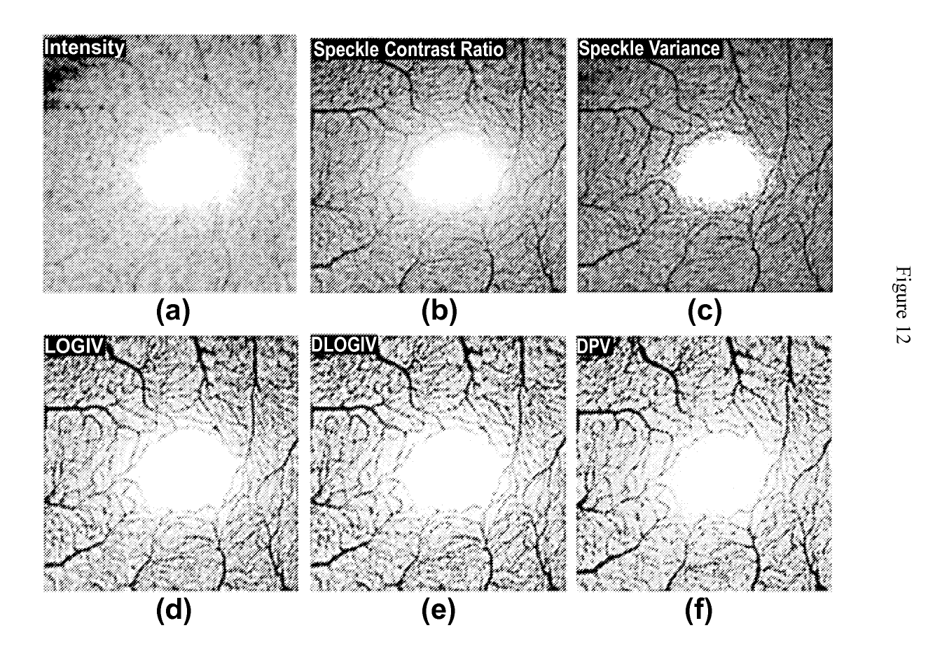

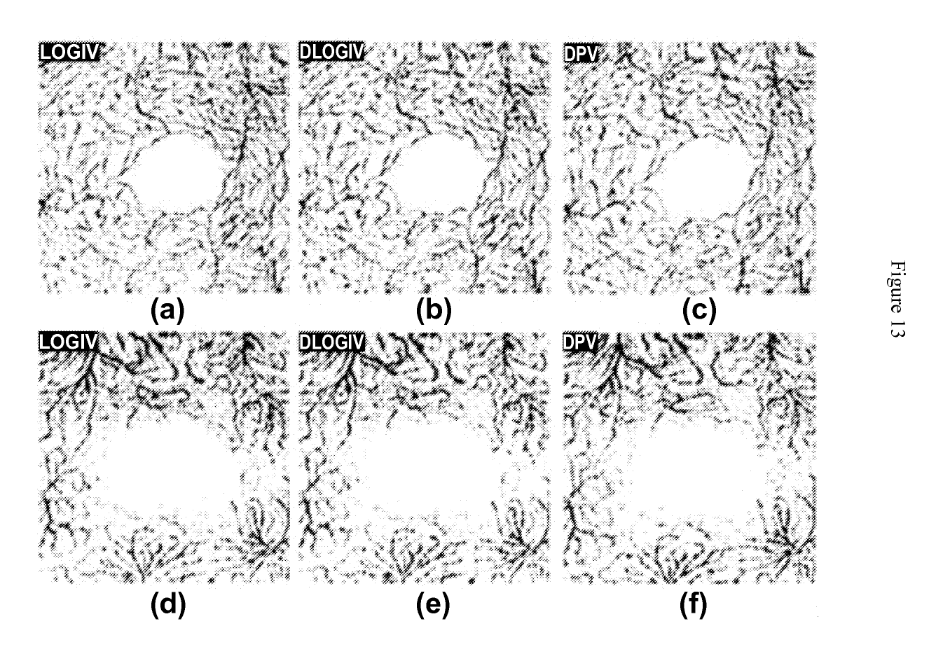

To create the retinal en face views, the inner/outer photoreceptor segments (IS/OS) and vitreoretinal interface were detected using a segmentation algorithm. Several depth integrated motion contrast en face images were generated by integrating the speckle variance, speckle contrast ratio, LOGIV, DLOGIV, and DPV between three different regions in the inner retina relative to IS/OS and vitreoretinal interface (FIGS. 12-13).

### Example 1

### Optical Coherence Angiography Using Logarithm of Intensity and Phase Contrast Imaging Methods

### Logarithmic Intensity Contrast (LOGIC) Imaging

Linear complex OCT signal is given by the following equation (Eq.) (1), where z, T, I, and φ are depth, time separation between two B-scans (measurements), linear intensity, and phase.

OCT Signal=√I(z,T)e^{jφ(z,T)} (Eq. 1)

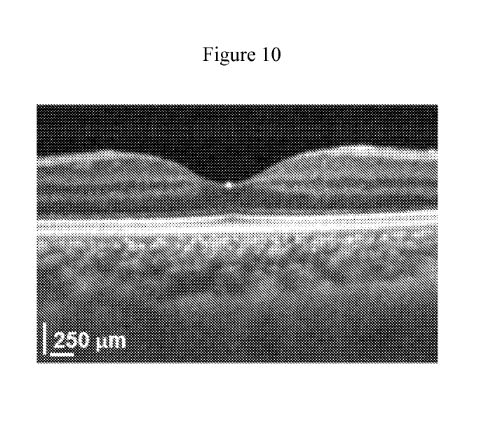

FIG. 10 depicts the conventional OCT intensity tomogram across the fovea centralis (5 mm) in logarithmic scale. While 2D tomogram (FIG. 10) can delineate the multiple retinal/choroidal layers, the microvasculature flow and the regions of motion may not be detected.

### 1. Logarithmic Intensity Contrast (LOGIC) Imaging

Multiple B-scans are acquired over the same transversal sample section. LOGIV is obtained by calculating logarithm of the intensity measurements (log(I⁽ⁱ⁾(z,T))) of the same transverse points (separated in time) and the statistical variance of logarithm of these intensities. To capture 3D motion contrast image, the same procedure is repeated for the neighboring B-scans. The following equation shows LOGIV contrast for a given position (x,y,z) in the sample, where i is the B-scan number.

$$\begin{matrix}Contrast = {σ}_{Log ( I ( x , y , z ) )}^{2} \\ = \frac{1}{N} {\sum }_{i = 1}^{i = N} ( log ( {I}^{( i )} ( x , y , z , T ) - \\ \frac{1}{N} {\sum }_{i = 1}^{i = N} {log ( {I}^{( i )} ( x , y , z , T ) )}^{2}\end{matrix} \qquad ( Eq . 2 )$$

### 2. Differential Logarithmic Intensity Contrast (DLOGIC) Imaging

Multiple B-scans are acquired over the same transversal sample section. DLOGIV is obtained by calculating the differences between two (or multiple) logarithm of the intensity measurements (log(I⁽ⁱ⁾(z,T))) of the same transverse points (separated in time) and the statistical variance of these logarithm of intensity changes. To capture 3D motion contrast image, the same procedure is repeated for the neighboring B-scans. The following equation shows logarithmic intensity differences and DLOGIV for a given position (x,y,z) in the sample, where i is the B-scan number.

$$\begin{matrix}Δ {LI}^{( i )} ( x , y , z , T ) = log ( {I}^{( i + 1 )} ( x , y , z , T ) ) - log ( {I}^{( i )} ( x , y , z , T ) ) & ( Eq . 3 ) \\ \begin{matrix} Contrast = {σ}_{Δ LI ( x , y , z )}^{2} \\ = \frac{1}{N - 1} {\sum }_{i = 1}^{i = N - 1} ( Δ {LI}^{( i )} ( x , y , z , T ) - \\ { \frac{1}{N - 1} {\sum }_{i = 1}^{i = N - 1} Δ {LI}^{( i )} ( x , y , z , T ) )}^{2}\end{matrix} & ( Eq . 4 )\end{matrix}$$

### 2D Tomogram and En Face View Visualization of the Retina Using Motion Contrast Imaging Methods

To study different motion contrast methods, four B-scans were acquired across the foveal centralis (2 mm). The averaged intensity of four obtained B-scans is depicted in FIG. 11(a). 2D speckle contrast ratio and speckle variance tomograms (FIGS. 11(b)-11(c)) show that these speckle contrast ratios capture not only regions of motion (between blue box) in the inner choroid and small vessels (white arrows) in the inner retina but also highly reflective stationary regions in IS/OS, RPE (between red box), and the inner retina. While the speckle variance (FIG. 11(c)) is able to capture the inner retina vessels (white arrow), it highlights the static regions of IS/OS and RPE (between redbox) as motion. Motion in the inner choroid is barely detected in this tomogram. FIGS. 11(d)-11(e) show the enhanced motion contrast in 2D LOGIV and DLOGIV tomograms. White static areas (between red boxes) captured in 2D speckle tomograms (FIGS. 11(b)-11(c)) are invisible in 2D LOGIV and DLOGIV tomograms (FIGS. 11(d)-11(e)). Regions of motion in the inner choroid (white band between blue boxes) and the small vessels in the inner retina (white arrows) are detectable in these 2D tomograms (FIGS. 11(d)-11(e)). To compare the intensity-based contrasts with DPV contrast, 2D DPV tomograms are shown in FIGS. 11(f)-11(g) before and after phase error correction and compensation, respectively. FIG. 11(f) demonstrate DPV is unable to capture motion without use of correction/compensation algorithms and an extra hardware module. In addition, the calibration mirror image limits imaging depth. Thus, the simplicity and motion sensitivity of LOGIV and DLOGIV may make these two contrast methods more attractive than other proposed phase- and linear intensity-based methods (DPV, speckle variance, and speckle contrast ratio) for capturing motion and microvasculature.

FIGS. 12(a)-12(f) illustrate the inverted intensity, speckle contrast ratio, speckle variance, LOGIV, DLOGIV, and DPV en face views generated by integrating their values between the region 30 μm posterior to the vitreoretinal interface and the region 130 μm anterior to IS/OS. FIG. 12(a) shows that the meshwork of capillaries is barely visible in the intensity en face view. Although small vessels and capillaries are seen in the speckle contrast ratio, speckle variance, en face images (FIGS. 12(b)-12(c)), the narrow dynamic range and high sensitivity to hyper-reflective static regions degrade retinal microvasculature enface visualization through contrast integration in the depth. Gray areas highlight the hyper-reflective stationary regions captured around the fovea avascular zone (FAZ) and between the interconnected microvasculature networks (FIGS. 12(b)-12(c)). Motion contrast enhancement is depicted in FIGS. 12(d)-12(e) using LOGIV and DLOGIV methods. Blood vessels in the ganglion cell layer and capillary meshwork of the inner plexiform layer are visualized in the LOGIV and DLOGIV en face views (FIGS. 12(d)-12(e)). FAZ is resolvable by considering the capillary network around it as shown in the LOGIV and DLOGIV images in FIGS. 12(d)-12(e). To compare retinal visualization using the proposed intensity-based motion contrast methods with the phase contrast method, the DPV en face image (FIG. 12(f)) is generated by summing DPVs over the same regions in the inner retina. Although LOGIV, DLOGIV, and DPV en face images (FIGS. 12(d)-12(f)) achieve the similar contrast for foveal vasculature visualization, DPV is a complicated method due to its need for the correction/compensation algorithms and an extra optical module.

To show the capillary meshwork of the inner retina through depth using logarithmic intensity-based motion contrast methods, the LOGIV and DLOGIV en face views are generated by integrating their values through different depths. FIGS. 13(a)-13(b) show the capillary network of the inner retina between the regions 255 μm and 216 μm anterior to IS/OS in the inverted LOGIV, and DLOGIV en face views. The inverted DPV en face view (FIG. 13(c)) depicts the similar capillary meshwork of the inner retina in the same region. Similar retinal microvasculature network is also detected between the regions 216 μm and 169 μm anterior to IS/OS (FIGS. 13(d)-13(f)) in the inverted LOGIV, DLOGIV, and DPV en face views. FIGS. 13(a)-13(f) clearly reveal depth-related variations of capillary meshwork morphology through the inner retina.

### 3. Joint Differential Intensity and Phase Contrast (JDIPC) Imaging

JDIPC is realized by calculating the differences between two (or multiple) logarithm of the received complex OCT signal measurements (log(OCT Signal⁽ⁱ⁾(z,T))) of the same transverse points (separated in time) and statistical analysis (such as covariance) between these phase and intensity changes (real and imaginary parts) after phase (or imaginary part) correction and compensation.

$$\begin{matrix} Log ( OCT Signal ) = 0.5 * log ( I ( z , T ) ) + j ( φ ( z , T ) ) & ( Eq . 5 ) \\ Δ LI {φ}^{( i )} ( z , T ) = 0.5 * \{ log ( {I}^{( i + 1 )} ( z , T ) ) - log ( {I}^{( i )} ( z , T ) ) \} + j \{ {φ}^{( i + 1 )} ( z , T ) - {φ}^{( i )} ( z , T ) \} = 0.5 * Δ {LI}^{( i )} {jΔφ}^{( i )} & ( Eq . 6 ) \\ \begin{matrix} Contrast = Cov \{ Δ LI \times Δ ϕ \} \\ = \frac{1}{2 ( N - 1 )} {\sum }_{i = 1}^{i = N - 1} \{ \begin{matrix}( Δ {LI}^{i} - ( \frac{{\sum }_{i = 1}^{i = N - 1} Δ {LI}^{i}}{N - 1} ) ) \times \\ ( Δ {ϕ}^{i} - ( \frac{{\sum }_{i = 1}^{i = N - 1} Δ {ϕ}^{i}}{N - 1} ) )\end{matrix} \}\end{matrix} & ( Eq . 7 )\end{matrix}$$

One important post-image processing is removing low signal region. Since the low signal-to-noise ratio exhibits random phase distribution, it disturbs flow images. Phase changes are masked for display by applying a particular threshold to the contrast. By decreasing transversal optical beam displacement for dense sampling, averaging and/or autocorrelation algorithm can be applied over a given spatial windows size for improving contrast.

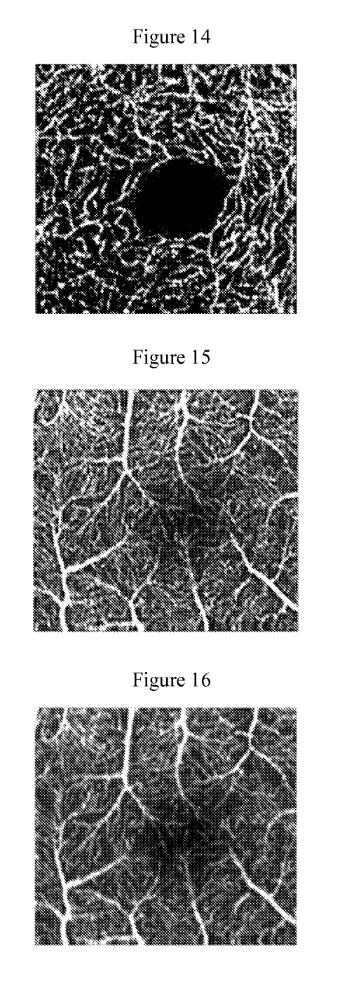

To perform JDIPC, four B-scans were acquired over the same transverse position (or slice). Time separations was T_{B}=5 ms between B-scans for capturing the same position, respectively. Four complex OCT signal were recorded over the same transverse point separated in time. JDIPC captured the differences between multiple complex logarithm of complex OCT signals of the same transverse points (separated in time) and calculated a statistical measure (such as covariance) of real and corrected imaginary parts. To measure and remove timing-induced imaginary part (phase) error due to the random delay between the trigger signal and the subsequent A-to-D conversion (sample clock), a calibration signal was generated using a stationary mirror in the calibration arm (FIG. 2). The calibration signal was located at a depth of 2 mm in the OCT intensity image. The corrected phase differences between adjacent B-scans for the same transverse point at a given depth were calculated by subtracting the phase difference of the calibration signal, linearly scaled with the sample signal depth, from the measured phase differences. Phase unwrapping was performed on all measurements. A weighted mean algorithm estimated and removed the bulk axial motion phase change error. The same described procedures were repeated for the adjacent transverse points in the same and neighboring B-scans to capture the retinal vasculature in 2D and 3D data sets. To remove SNR-limited intensity and phase change errors in 2D and 3D data sets for vasculature visualization, an average intensity threshold (10 dB above the mean value of the noise floor) was applied; all contrasts with average intensity values<mean (log₁₀(Iₙₒᵢₛₑ))+10 dB were set to zero in the corresponding images (FIG. 14). To create the retinal en face views, the inner/outer photoreceptor segments (IS/OS) and vitreoretinal interface were detected using a segmentation algorithm. The depth integrated motion contrast en face image was generated by integrating JDIPC between the regions 255 μm and 216 μm anterior to IS/OS in the JDIPC en face view (FIG. 14). Using JDIPC method, foveal avascular zone (FAZ) is resolvable by detecting the capillary network around it as shown in the JDIPC image in FIG. 14.

### Example 2

### Optical Coherence Angiography Using Generalized Intensity and Differential Phase Contrast Imaging Methods

### Generalized Intensity and Differential Phase Contrast (GIDPC) Imaging

Two different approaches are demonstrated for GIDPC:

(a) A new variable is defined and given by the following function

H=H(I,Δφ) (Eq. 8)

We propose to calculate the n^{th} moment of a new random variable (H) about a deterministic value of c (c can be mean of H (=E{H})). E is the expectation operator. The generalized form of contrast is given by:

Contrast=E{[H−c]ⁿ} (Eq. 9)

Thus first order contrast or second order contrast can be expressed as

Contrast⁽¹⁾=E{H} (Eq. 10)

Contrast⁽²⁾=E{H²}−E{H}² (Eq. 11)

where I and Δφ are linear intensity and differential phase measurements.

Multiple B-scans are acquired over the same transversal sample section. GIDPC is obtained by recording two (or multiple) linear intensities, calculating the differences between two (or multiple) phase measurements (Δφ⁽ⁱ⁾(x,y,z,T)=φ⁽ⁱ⁾(x,y,z,T)−φ⁽ⁱ⁻¹⁾(x,y,z,T)) of the same transverse points (separated in time), and computing the statistical n^{th} moment of “H(I, Δφ)” around a value c such as E{H(I, Δφ)}. In order to capture 3D image, neighboring B-scans are captured. The same method is applied to obtain 2D contrast images for neighboring B-scans. For example, H and contrast can be given by:

H⁽ⁱ⁾=log(I⁽ⁱ⁾(x,y,z,T))+{φ⁽ⁱ⁺¹⁾(x,y,z,T)−φ⁽ⁱ⁾(x,y,z,T)}=log(I⁽ⁱ⁾(x,y,z,T))+Δφ⁽ⁱ⁾(x,y,z,T) (Eq. 12)

Contrast=E{H²}−E{H}²=E{(log(I(x,y,z))+Δφ(x,y,z))²}−E{log(I(x,y,z))+Δφ(x,y,z)}² (Eq. 13)

Contrast=σ²_{log(I)}+σ²_{Δφ}−2cov(log(I),Δφ) (Eq. 14)

Equation (12) shows the defined random variable “H(a,b)=log(a)+b” in terms of intensity and the differential phase for a given position (x,y,z) in the sample, where i is the B-scan number.

(b) Two new variables are defined and given by the following functions

G₁=G₁(I) (Eq. 15)

G₂=G₂(Δφ) (Eq. 16)

We propose to calculate the n^{th} and m^{th} moments of new random variables (G₁ and G₂) about two deterministic values of c₁ and c₂ (cᵢ can be means of Gᵢ (=E{Gᵢ}, i=1,2), respectively. The generalized form of contrast is given by

Contrast=K(E{[G₁−c₁]ⁿ},E{[G₂−c₂]^{m}}) (Eq. 17)

where K is a function of two variables.

Multiple B-scans are acquired over the same transversal sample section. GIDPC is obtained by recording two (or multiple) linear intensities, calculating the differences between two (or multiple) phase measurements (Δφ⁽ⁱ⁾(x,y,z,T)=φ⁽ⁱ⁾)(x,y,z,T)−φ⁽ⁱ⁻¹⁾(x,y,z,T)) of the same transverse points (separated in time), and computing the statistical n^{th} and M^{th} moments of G₁ and G₂ around two values of c₁ and c₂. In order to capture 3D image, neighboring B-scans are captured. The same method is applied to 2D obtain contrast images for neighboring B-scans. For example, G₁, G₂, and contrast can be given by:

G₁⁽ⁱ⁾=log(I⁽ⁱ⁾(x,y,z,T)) (Eq. 18)

G₂⁽ⁱ⁾={φ⁽ⁱ⁺¹⁾(x,y,z,T)−φ⁽ⁱ⁾(x,y,z,T)}=Δφ⁽ⁱ⁾(x,y,z,T) (Eq. 19)

Contrast=E{[G₁−E{G₁}]²}+E{[G₂−E{G₂}]²}=σ²_{log(I)}+σ²_{Δφ} (Eq. 20)

where K(a,b)=a+b;

To perform GIDPC-b, four B-scans were acquired over the same transverse position (or slice). Time separations was T_{B}=5 ms between B-scans for capturing the same position, respectively. Four complex OCT signal were recorded over the same transverse point separated in time. GIDPC-b captured multiple logarithm intensities and the differences between successive phase measurements of the same transverse points (separated in time) and calculated the motion contrast using the given flowchart in FIG. 7. To measure and remove timing-induced phase error due to the random delay between the trigger signal and the subsequent A-to-D conversion (sample clock), a calibration signal was generated using a stationary mirror in the calibration arm (FIG. 2). The calibration signal was located at a depth of 2 mm in the OCT intensity image. The corrected phase differences between adjacent B-scans for the same transverse point at a given depth were calculated by subtracting the phase difference of the calibration signal, linearly scaled with the sample signal depth, from the measured phase differences. Phase unwrapping was performed on all measurements. A weighted mean algorithm estimated and removed the bulk axial motion phase change error. The same described procedures were repeated for the adjacent transverse points in the same and neighboring B-scans to capture the retinal vasculature in 2D and 3D data sets. To remove SNR-limited intensity and phase change errors in 2D and 3D data sets for vasculature visualization, an average intensity threshold (10 dB above the mean value of the noise floor) was applied; all contrasts with average intensity values<mean (log₁₀(Iₙₒᵢₛₑ))+10 dB were set to zero in the corresponding images (FIG. 15). To create the retinal en face views, the inner/outer photoreceptor segments (IS/OS) and vitreoretinal interface were detected using a segmentation algorithm. The depth integrated motion contrast en face image (FIG. 15) was generated by integrating GIDPC-b between by integrating their values between the region 30 μm posterior to the vitreoretinal interface and the region 130 μm anterior to IS/OS.

### Generalized Intensity Ratio and Differential Phase Contrast (GIRDPC) Imaging

Applicants propose two different methods using intensity ratios and differential phases. In order to obtain these contrasts, multiple B-scans are acquired over the same transversal sample section. Intensity ratios and differential phases are obtained by calculating two (or multiple) linear intensity ratios (RI⁽ⁱ⁾(x,y,z,T)=I⁽ⁱ⁾(x,y,z,T)/I⁽ⁱ⁻¹⁾(x,y,z,T)) and the differences between two (or multiple) phase measurements (Δφ⁽ⁱ⁾(x,y,z,T)=Δφ⁽ⁱ⁾(x,y,z,T)−Δφ⁽ⁱ⁻¹⁾(x,y,z,T)) of the same transverse points (separated in time). The same methods developed for GIDPC in (a) and (b) are used for generating GIRDPC by replacing intensity (I) with ratio of two successive intensity measurements (RI⁽ⁱ⁾(x,y,z,T)=I⁽ⁱ⁾(x,y,z,T)/I⁽ⁱ⁻¹⁾(x,y,z,T))). Therefore,

a—The defined variable is given by the following function:

H=H(RI,Δφ) (Eq. 21)

Applicants propose to calculate the n^{th} moment of a new random variable (H) about a deterministic value of c (c can be mean of H(=E{H})). The generalized form of contrast is given by:

Contrast=E{[H−c]ⁿ} (Eq. 22)

Thus first order contrast or second order contrast can be expressed as

Contrast⁽¹⁾=E{H} (Eq. 23)

Contrast⁽²⁾=E{H²}−E{H}² (Eq. 24)

where RI and Δφ are linear intensity ratio and differential phase measurement. For example, H and contrast can be given by:

H⁽ⁱ⁾=log(I⁽ⁱ⁺¹⁾(x,y,z,T)/I⁽ⁱ⁾(x,y,z,T))+{φ⁽ⁱ⁺¹⁾(x,y,z,T)−φ⁽ⁱ⁾(x,y,z,T)}=log(I⁽ⁱ⁺¹⁾(x,y,z,T)−log(I⁽ⁱ⁾(x,y,z,T))+Δφ⁽ⁱ⁾(x,y,z,T)=Δ log(I⁽ⁱ⁾(x,y,z,T))+Δφ⁽ⁱ⁾(x,y,z,T) (Eq. 25)

Contrast=E{H²}−E{H}²=E{(Δ log(I(x,y,z))+Δφ(x,y,z))²}−E{Δ log(I(x,y,z))+Δφ(x,y,z)}² (Eq. 26)

Contrast=σ²_{Δ log(I)}+σ²_{Δφ}−2cov(Δ log(I),Δφ) (Eq. 27)

b—Two new variables are defined and given by the following functions

G₁=G₁(RI) (Eq. 28)

G₂=G₂(Δφ) (Eq. 29)

Applicants propose to calculate the n^{th} and m^{th} moments of new random variables (G₁ and G₂) about two deterministic values of c₁ and c₂ (cᵢ can be means of Gᵢ (=E{Gᵢ}, i=1,2), respectively. The generalized form of contrast is given by:

Contrast=K(E{[G₁−c₁]ⁿ},E{[G₂−c₂]^{m}}) (Eq. 30)

where K is a function of two variables.

For example, G₁, G₂, and contrast can be given by

G₁⁽ⁱ⁾=log(I⁽ⁱ⁺¹⁾(x,y,z,T)/I⁽ⁱ⁾(x,y,z,T))=log(I⁽ⁱ⁺¹⁾(x,y,z,T)−log(I⁽ⁱ⁾(x,y,z,T))=Δ log(I⁽ⁱ⁾(x,y,z,T)) (Eq. 31)

G₂⁽ⁱ⁾={φ⁽ⁱ⁺¹⁾(x,y,z,T)−φ⁽ⁱ⁾(x,y,z,T)}=Δφ⁽ⁱ⁾(x,y,z,T) (Eq. 32)

Contrast=E{[G₁−E{G₁}]²}+E{[G₂−E{G₂}]²}=σ²_{Δ log(I)}+σ²_{Δφ} (Eq. 33)

where K(a,b)=a+b.

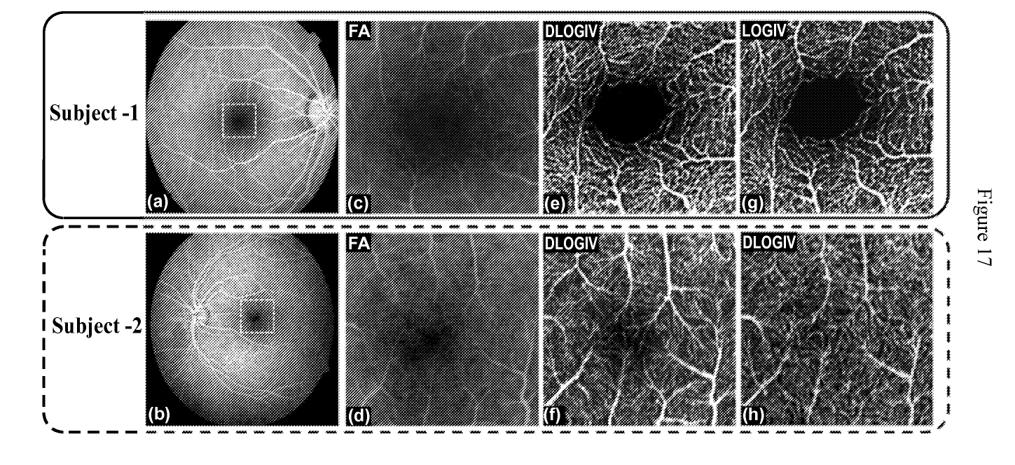

To perform GIRDPC-b, four B-scans were acquired over the same transverse position (or slice). Time separations was T_{B}=5 ms between B-scans for capturing the same position, respectively. Four complex OCT signal were recorded over the same transverse point separated in time. GIRDPC-b captured multiple ratios of intensities between successive measurements ratios and the differences between successive phase measurements of the same transverse points (separated in time) and calculated the motion contrast using the given flowchart in FIG. 9. To measure and remove timing-induced phase error due to the random delay between the trigger signal and the subsequent A-to-D conversion (sample clock), a calibration signal was generated using a stationary mirror in the calibration arm (FIG. 2). The calibration signal was located at a depth of 2 mm in the OCT intensity image. The corrected phase differences between adjacent B-scans for the same transverse point at a given depth were calculated by subtracting the phase difference of the calibration signal, linearly scaled with the sample signal depth, from the measured phase differences. Phase unwrapping was performed on all measurements. A weighted mean algorithm estimated and removed the bulk axial motion phase change error. The same described procedures were repeated for the adjacent transverse points in the same and neighboring B-scans to capture the retinal vasculature in 2D and 3D data sets. To remove SNR-limited intensity and phase change errors in 2D and 3D data sets for vasculature visualization, an average intensity threshold (10 dB above the mean value of the noise floor) was applied; all contrasts with average intensity values<mean (log₁₀(Iₙₒᵢₛₑ))+10 dB were set to zero in the corresponding images (FIG. 16). To create the retinal en face views, the inner/outer photoreceptor segments (IS/OS) and vitreoretinal interface were detected using a segmentation algorithm. The depth integrated motion contrast en face image (FIG. 16) was generated by integrating GIRDPC-b between by integrating their values between the region 30 μm posterior to the vitreoretinal interface and the region 130 μm anterior to IS/OS.

To compare DLOGIV and LOGIV methods with FA, OCT and FA were performed on two normal subjects. En face LOGIV and DLOGIV images were capable of capturing the microvasculature through depth. The sensitivity and resolution of parafoveal capillary meshwork images from both DLOGIV and LOGIV were significantly greater than FA images of the same regions (FIG. 17). While DLOGIV, LOGIV and FA captured and quantified FAZs in one eye of one healthy subject (FIGS. 17(c,e,g)), no FAZ was discernible in either eye of the other healthy subject (FIGS. 17(d,f,h)). We could prove the feasibility of a novel imaging methods (LOGIV and DLOGIV) for non-invasive, dye-free visualization and quantification of the retinal microvasculature using a SS-OCT at 1060 nm. Compared to DPV, LOGIV and DLOGIV does not rely on phase information. Therefore, it is less sensitive to the phase instability of the system and environment, and there is no need for phase correction/compensation algorithms and additional optical modules. As such, DLOGIV may be advantageous to both DPV and invasive FA for imaging the retinal microvasculature and be a helpful diagnostic tool in the future.

### Example 3

### Optical Angiography Using Logarithmic Intensity and Differential Intensity Imaging Methods

Applicants propose two noninvasive methods for vasculature visualization. These methods are simple and cheap using a CCD camera and a fundus illuminator. Scanning tool is replaced by a solid state camera such as a CCD camera and a fundus illuminator. This method is able to capture vasculature over wide field of view using a CCD camera. Although these methods may not provide depth information, they don't need coherence gating for capturing retina images. The proposed methods are applicable for not only tissue (retina, choroid, etc.) vasculature visualization but also detecting mobility in a structure.

### Method

A fast CCD (charge coupled device) (for example: exposure time<1 ms) and a fundus illumination (visible or near infrared wavelength range) are used to image sample (tissue, retina, etc.). Several images (N en face retina images) are obtained in T milliseconds range (varies between 50 milliseconds to 1 second). This procedure can be repeated multiple times (M). M sets of N en face retina images are acquired. In order to capture an image of the vasculature, two different methods are demonstrated:

### 1. Logarithmic Intensity Contrast Imaging

En face intensity image (I⁽ⁱ⁾(x,y,T)) is generated by collecting data from CCD camera at a given time point (tᵢ). CCD size and pixel numbers determine the transverse resolution of the proposed methods for capturing vasculature. N successive en face images are obtained in N*tᵢ seconds. Time separation is tᵢ−tᵢ₋₁=T. This set of data contains N en face images. The same procedure is applied to capture sample (retina) images multiple times (other M−1 sets). Logarithm of en face intensity images are generated for M*N subsets (log(I^{(i,j)}(x,y,T)). i and j are the en face image number in a given set and set number, respectively. (1≦i≦N and 1≦j≦M)

After image registration, the n^{th} moment of each data set (log(I^{(i,j)}(x,y,T)) is calculated about a deterministic value of c (c can be mean of that data set (=E{log(I^{(i,j)}(x,y,T)})). E is the expectation operator. For example (n=2, second moment), contrast can be given for the j^{th} set by

H^{(i,j)}=log(I^{(i,j)}(x,y,T)) (Eq. 34)

Contrast^{(j)}=E{H^{(i,j)2}}−E{H^{(i,j)}}²=E{(log(I^{(i,j)}((x,y,z)))²}−E{log(I^{(i,j)}((x,y,z)))}²=σⱼ²_{log(I)} (Eq. 35)

To improve contrast, we sum all the calculated contrasts

Improved Contrast=Σⱼ₌₁^{M}σ_{j log(I)}² (Eq. 36)

FIG. 18 shows a simple flowchart representing the required procedures for vasculature visualization using logarithmic intensity method.

### 2. Differential Logarithmic Intensity Contrast Imaging

En face intensity image (I⁽ⁱ⁾(x,y,T)) is generated by collecting data from a CCD at a given time point (tᵢ). CCD size and pixel numbers determine the transverse resolution of the proposed method for capturing vasculature. N successive en face images are obtained in N*tᵢ seconds. Time separation is tᵢ−tᵢ₋₁=T. This set of data contains N en face images. N successive en face images are obtained in N*tᵢ seconds. Time separation is tᵢ−tᵢ₋₁=T. This set of data contains N en face images. The same procedure is applied to capture sample (retina) images multiple times (other M−1 sets). Logarithm of en face intensity images are generated for M*N subsets (log(I^{(i,j)}(x,y,T)). i and j are the en face number in a given set and set number, respectively. (s1≦i≦N and 1≦j≦M).

After image registration, differences between successive logarithmic en face images in each set are generated.

D^{(i-1,j)}=log(I^{(i,j)}(x,y,T))−log(I^{(i-1,j)}(x,y,T) (Eq. 37)

For example (n=2, second moment), contrast can be given for the j^{th} set by

Contrast^{(j)}=E{D^{(i-1,j)2}}−E{D^{(i-1,j)}}²=E{(log(I^{(i,j)}(x,y,T))−log(I^{(i-1,j)}(x,y,T)))²}−E{log(I^{(i,j)}(x,y,T))−log(I^{(i-1,j)}(x,y,T))}²=σⱼ²_{Δ log(I)} (Eq. 38)

To improve contrast, we sum all the calculated contrasts

Improved Contrast=Σⱼ₌₁^{M}σ_{jΔ log(I)}² (Eq. 39)

Applicants are also able to capture vasculature by calculating intensity ratios between successive en face images (I^{(i,j)}(x,y,T)/I^{(i-1,j)}(x,y,T)). In order to do that, we need to replace D^{(i-1,j)} with (I^{(i,j)}(x,y,T)/I^{(i-1,j)}(x,y,T)) in (Eq. 38) and (Eq. 39).

FIG. 19 shows a simple flowchart representing the required procedures for vasculature visualization using the differential logarithmic intensity method. In both proposed methods, Applicants can replace logarithm with other functions such as hyperbolic functions to capture vasculature. These two proposed methods are able to capture retinal and choroidal vasculature using short wavelength (green light) and long wavelength (red light), respectively. Red blood cells absorb green light and green light is highly absorbed and scattered by the RPE. Thus, en face image data collected with the green light will capture the retinal vasculature preferentially. Red light is less scattered and absorbed by the layers in the retina and by the RPE, and thus can pass through to capture images of the deeper choroidal vessels permitting the technique to map the choroidal vasculature.

1. A method for ascertaining motion contrast in a sample using an optical coherence tomography system comprising:
(i) acquiring multiple B-scans of the sample separated in time over the same transverse position using optical coherence tomography (OCT), wherein each of the B-scans comprise data acquired during multiple A-scans over a range of transverse locations;
(ii) acquiring multiple OCT intensity (I) measurements based on the data of the B-scans over the same transverse point separated in time;
(iii) ascertaining logarithms of the OCT intensity measurements over the same transverse point separated in time;
(iv) ascertaining motion contrast based upon the variance of logarithmic intensity measurements of the same transverse point acquired in the successive B-scans separated in time; and
(v) repeating the same described procedures (i-iv) for the adjacent transverse points in the same and neighboring B-scans to ascertain motion contrast in the sample.

2. The method of claim 1, wherein motion contrast based on the variance of the measured logarithm intensities in the successive B-scans is ascertained according to Equation 2.

3. The method of claim 1, wherein motion contrast based on the variance of differences of the logarithm intensities between the successive B-scans is ascertained according to Equation 4.

4. A method for ascertaining motion contrast in a sample, comprising:
(i) acquiring multiple B-scans separated in time over the same transverse position using OCT;
(ii) acquiring multiple complex OCT signals based on the B-scans over the same transverse point separated in time;
(iii) ascertaining complex logarithms of the complex OCT signals over the same transverse point separated in time;
(iv) ascertaining differences between the successive calculated complex logarithms for the same transverse point;
(v) ascertaining an statistical measure (covariance) between the real and corrected and compensated imaginary parts of the complex logarithm differences for the same transverse point;
(vi) ascertaining the motion contrast based on the statistical measure (covariance); and
(vii) repeating the same described procedures (i-vi) for the adjacent transverse points in the same and neighboring B-scans to ascertain motion contrast in the sample.

5. The method of claim 4, wherein:
(i) the complex OCT signals based on the B-scans are acquired according to Equation 1;
(ii) the complex logarithms of the complex OCT signals based on the B-scans are ascertained according to Equation 5;
(iii) the differences between the corrected and compensated complex logarithms are ascertained according to Equation 6; and
(iv) the motion contrast is ascertained according to Equation 7.

6. The method of claim 1, wherein the variance of logarithm intensity is ascertained independent of OCT phase data.

7. A method for ascertaining motion contrast in a sample using an OCT system, comprising:
(i) acquiring multiple B-scans separated in time over the same transverse position using OCT;
(ii) acquiring multiple OCT intensity (I) measurements based on the B-scans over the same transverse point separated in time;
(iii) acquiring multiple OCT phase measurements based on the B-scans over the same transverse point separated in time;
(iv) ascertaining corrected and compensated differences between the successive OCT phase measurements (Δφ) for the same transverse point separated in time;
(v) ascertaining a variable h according to: h=H(I,Δφ); where H denotes a function I and Δφ;
(vi) ascertaining a n^{th} moment of the variable h about a deterministic value of c, wherein n is an integer;
(vii) ascertaining the motion contrast based on the n^{th} moment; and
(viii) repeating the same described procedures for the adjacent transverse points in the same and neighboring B-scans to ascertain motion contrast in the sample.

8. The method of claim 7, wherein:
(i) the deterministic value of c is the mean of h;
(ii) n=2;
(iii) H(a,b)=log(a)+b; and
(iii) the motion contrast is ascertained according to Equation 14.

9. A method for ascertaining motion contrast in a sample, comprising:
(i) acquiring multiple B-scans separated in time over the same transverse position using OCT;
(ii) acquiring multiple OCT intensity measurements (I) based on the B-scans over the same transverse point separated in time;
(iii) ascertaining a variable g₁ according to: g₁=G₁(I); where G₁ denotes a function of variable I;
(iv) ascertaining a n^{th} moment of the variable g₁ about a deterministic value of c₁, wherein n is an integer;
(v) acquiring multiple OCT phase measurements based on the B-scans over the same transverse point separated in time;
(vi) ascertaining corrected and compensated differences between the OCT phase measurements (Δφ) for the same transverse point separated in time;
(vii) ascertaining a variable g₂ according to: g₂=G₂(Δφ); where G₂ denotes a function of Δφ;
(viii) ascertaining a m^{th} moment of the variable g₂ about a deterministic value of c₂, wherein m is an integer;
(ix) ascertaining a variable k according to: k=K(n^{th} moment of the variable g₁ about a deterministic value of c₁, m^{th} moment of the variable g₂ about a deterministic value of c₂), wherein m and n are integers and K denotes a function of two variables;
(x) ascertaining the motion contrast based on the variable k; and
(xi) repeating the same described procedures for the adjacent transverse points in the same and neighboring B-scans to ascertain motion contrast in the sample.

10. The method of claim 9, wherein:
(i) G₁(x)=log(x);
(ii) G₂(y)=y;
(iii) n=m=2;
(iv) the deterministic values of c1 and c2 are the mean of g1 and g2, respectively.
(v) k=K(a,b)=a+b; and
(vi) the motion contrast is ascertained according to Equation 20.

11. A method for ascertaining motion contrast in a sample, comprising:
(i) acquiring multiple B-scans separated in time over the same transverse position using OCT;
(ii) acquiring multiple OCT intensity (I) measurements based on the B-scans over the same transverse point separated in time;
(iii) ascertaining linear intensity ratios (RIs) between the successive OCT intensity measurements for the same transverse point;
(iv) acquiring multiple OCT phase measurements based on the B-scans over the same transverse point separated in time;
(v) ascertaining corrected and compensated differences between the successive OCT phase measurements (Δφ) for the same transverse point separated in time;
(vi) ascertaining a variable h according to: h=H(RI,Δφ); where H denotes a function of RI and Δφ;
(vii) ascertaining a n^{th} moment of the variable h about a deterministic value of c, wherein n is an integer;
(viii) ascertaining the motion contrast based on the n^{th} moment; and
(ix) repeating the same described procedures for the adjacent transverse points in the same and neighboring B-scans to ascertain motion contrast in the sample.

12. The method of claim 11, wherein:
(i) the deterministic value of c is the mean of h;
(ii) m=n=2;
(iii) H(a,b)=log(a)+b;
(iv) the motion contrast is ascertained according to Equation 27.

13. A method for ascertaining motion contrast in a sample, comprising:
(i) acquiring multiple B-scans separated in time over the same transverse position using OCT;
(ii) acquiring multiple OCT intensity measurements (I) based on the B-scans over the same transverse point separated in time;
(iii) ascertaining linear intensity ratios (RIs) between the successive OCT intensity measurements for the same transverse point;
(iv) ascertaining a variable g1 according to: g₁=G₁(RI); where G₁ denotes a function of variable RI;
(v) ascertaining a n^{th} moment of the variable g₁ about a deterministic value of c₁, wherein n is an integer;
(vi) acquiring multiple OCT phase measurements based on the B-scans over the same transverse point separated in time;
(vii) ascertaining corrected and compensated differences between the OCT phase measurements (Δφ) for the same transverse point separated in time;
(viii) ascertaining a variable g₂ according to: g₂=G₂(Δφ); where G₂ denotes a function of variable Δφ;
(ix) ascertaining a m^{th} moment of the variable g₂ about a deterministic value of c₂, wherein m is an integer;
(x) ascertaining a variable k according to: k=K(n^{th} moment of the variable g₁ about a deterministic value of c₁, M^{th} moment of the variable g₂ about a deterministic value of c₂), wherein m and n are integers and K denotes a function of two variables;
(xi) ascertaining the motion contrast based on the variable k; and
(xii) repeating the same described procedures for the adjacent transverse points in the same and neighboring B-scans to ascertain motion contrast in the sample.

14. The method of claim 13, wherein:
(i) G1(x)=log(x);
(ii) G2(y)=y;
(iii) n=m=2;
(iv) the deterministic values of c₁ and c₂ are the mean of g₁ and g₂, respectively.
(v) k=K(a,b)=a+b; and
(vi) the motion contrast is ascertained according to Equation 33.

15. A method for ascertaining motion contrast in a sample, comprising:
(i) acquiring multiple B-scans separated in time over the same transverse position using OCT;
(ii) acquiring multiple complex OCT signals based on the B-scans over the same transverse point separated in time;
(iii) ascertaining complex OCT signal ratios (RCSs) between the successive OCT signal measurements for the same transverse point;
(iv) ascertaining a variable g₁ according to: g₁=G₁([abs(RCS)]²); where G₁ denotes a function of variable of [abs(RCS)]²;
(v) ascertaining a n^{th} moment of the variable g₁ about a deterministic value of c₁, wherein n is an integer;
(vi) ascertaining a variable g₂ according to: g₂=G₂ (corrected and compensated angle(RCS); where G2 denotes a function of corrected and compensated variable of angle (RCS);
(viii) ascertaining a m^{th} moment of the variable g₂ about a deterministic value of c₂, wherein m is an integer;
(ix) ascertaining a variable k according to: k=K(n^{th} moment of the variable g₁ about a deterministic value of c₁, m^{th} moment of the variable g₂ about a deterministic value of c₂), wherein m and n are integers and K denotes a function of two variables;
(x) ascertaining the motion contrast based on the variable k; and
(xi) repeating the same described procedures for the adjacent transverse points in the same and neighboring B-scans to ascertain motion contrast in the sample.

16. The method of claim 15, wherein:
(i) G1(x)=log(x);
(ii) G2(y)=y;
(ii) n=m=2;
(iv) the deterministic values of c₁ and c₂ are the mean of g₁ and g₂, respectively.
(v) k=K(a,b)=a+b; and
(vi) the motion contrast is ascertained according to Equation 33.

17. The method of claim 1, wherein the motion contrast is ascertained by acquiring multiple B-scans separated in time using:
(i) a beam illumination in the sample arm of OCT system which scans the same transverse position multiple times; or
(ii) multiple coded frequency or polarization beam illuminations separated in time in the sample arm of a single or multiple OCT system which scan the same transverse position one (or multiple) times.

18. A method for ascertaining motion contrast in a sample, comprising:
(i) acquiring a set of N images of the sample using a digital camera and fundus illuminator;
(ii) acquiring a set of N intensity measurements (I) based on the set of N images;
(iii) ascertaining a set of N logarithms (log I) based on the set of N intensity measurements;
(iv) ascertaining a n^{th} moment of the set of N logarithms about a deterministic value of c; and
(v) ascertaining the motion contrast based on the n^{th} moment, wherein n and N are integers.

19. A method for ascertaining motion contrast in a sample, comprising:
(i) acquiring a set of N images of the sample using a digital camera and fundus illuminator;
(ii) acquiring a set of N intensity measurements (I) based on the set of N images;
(iii) ascertaining a set of N logarithms (log I) based on the set of N intensity measurements;
(iv) ascertaining a n^{th} moment of the set of N logarithms about a deterministic value of c;
(v) acquiring M n^{th} moments by repeating the steps of (i)-(iv) M times; and
(vi) ascertaining the motion contrast based on the sum of the M n^{th} moments, wherein M, N and n are integers.

20. The method of claim 18, wherein:
(i) the deterministic value of c is the mean of the set of N logarithms;
(ii) the n^{th} moment=E{[log I−c]ⁿ}; and
(iii) the motion contrast is ascertained according to Equation 35 or 36 for n=2.

21. A method for ascertaining motion contrast in a sample, comprising:
(i) acquiring a set of N images of the sample using a digital camera and fundus illuminator;
(ii) acquiring a set of N intensity measurements (I) based on the set of N images;
(iii) ascertaining a set of N logarithms (log I) based on the set of N intensity measurements;
(iv) ascertaining a set of N−1 logarithm differences (Δ log I) between two successive logarithms based on the set of N logarithms;
(v) ascertaining a n^{th} moment of the set of N−1 logarithm differences about a deterministic value of c; and
(vi) ascertaining the motion contrast based on the n^{th} moment, wherein n and N are integers.

22. A method for ascertaining motion contrast in a sample, comprising:
(i) acquiring a set of N images of the sample using a digital camera and fundus illuminator;
(ii) acquiring a set of N intensity measurements (I) based on the set of N images;
(iii) ascertaining a set of N logarithms (log I) based on the set of N intensity measurements;
(iv) ascertaining a set of N−1 logarithm differences (Δ log I) between two successive logarithms based on the set of N logarithms;
(v) ascertaining a n^{th} moment of the set of N−1 logarithm differences about a deterministic value of c;
(vi) acquiring M n^{th} moments by repeating the steps of (i)-(v) M times; and
(vii) ascertaining the motion contrast based on the sum of the M n^{th} moment, wherein M, N and n are integers.

23. The method of claim 21, wherein:
(i) the deterministic value of c is the mean of the set of N−1 logarithm differences;
(ii) the n^{th} moment=E{[Δ log I−c]ⁿ}; and
(iii) the motion contrast is ascertained according to Equation 38 or 39 for n=2.

24. A method for ascertaining motion contrast in a sample, comprising:
(i) acquiring a set of N images of the sample using a digital camera and fundus illuminator;
(ii) acquiring a set of N intensity measurements (I) based on the set of N images;
(iii) ascertaining a set of N−1 intensity ratios (RI) between two successive intensity measurements based on the set of N intensity measurements;
(iv) ascertaining a n^{th} moment of the set of N−1 intensity ratios about a deterministic value of c; and
(v) ascertaining the motion contrast based on the n^{th} moment, wherein n and N are integers.

25. A method for ascertaining motion contrast in a sample, comprising:
(i) acquiring a set of N images of the sample using a digital camera and fundus illuminator;
(ii) acquiring a set of N intensity measurements (I) based on the set of N images;
(iii) ascertaining a set of N−1 intensity ratios (RI) between two successive intensity measurements based on the set of N intensity measurements;
(iv) ascertaining a n^{th} moment of the set of N−1 intensity ratios about a deterministic value of c;
(v) acquiring M n^{th} moments by repeating the steps of (i)-(iv) M times; and
(vi) ascertaining the motion contrast based on the sum of the M n^{th} moment, wherein n, N and M are integers.

26. The method of claim 24, wherein:
(i) the deterministic value of c is the mean of the set of N−1 intensity ratios; and
(ii) the n^{th} moment=E{[RI−c]ⁿ}.

27. The method of claim 18, wherein the digital camera is a charge coupled device (CCD) or a complementary metal oxide semiconductor (CMOS) camera.

28. A method for detecting motion in a sample, comprising:
(i) ascertaining motion contrast in the sample according to the method of claim 1; and
(ii) detecting the motion in the sample based on the motion contrast.

29. A method for diagnosing/treating a disease in an individual, comprising:
(i) detecting motion in an area of the individual according to method 28; and
(ii) diagnosing/treating the disease in the individual based on the detected motion.

30. A method for visualizing vasculature in a sample, comprising:
(i) ascertaining motion contrast in the sample according to the method of claim 1; and
(ii) visualizing the vasculature based on the motion contrast.

31. A computer readable medium having computer executable instructions for ascertaining motion contrast in a sample according to the method of claim 1.

32. An OCT system comprising a computer readable medium having computer executable instruction for ascertaining motion contrast in a sample according to the method of claim 1.

33. The method of claim 19, wherein:
(i) the deterministic value of c is the mean of the set of N logarithms;
(ii) the n^{th} moment=E{[log I−c]ⁿ}; and
(iv) the motion contrast is ascertained according to Equation 35 or 36 for n=2.

34. The method of claim 22, wherein:
(i) the deterministic value of c is the mean of the set of N−1 logarithm differences;
(ii) the n^{th} moment=E{[Δ log I−c]ⁿ}; and
(iii) the motion contrast is ascertained according to Equation 38 or 39 for n=2.

35. The method of claim 25, wherein:
(i) the deterministic value of c is the mean of the set of N−1 intensity ratios; and
(ii) the n^{th} moment=E{[RI−c]ⁿ}.
